(19) United States
(12) Patent Application Publication
(10) Pub. No.: US 2024/0074947 A1
(43) Pub. Date: Mar. 7, 2024

(54) NONWOVEN FABRIC FOR SKIN CARE PRODUCTS, FACE MASK AND CLEANSING SHEET

(71) Applicant: Toray Industries, Inc., Tokyo (JP)

(72) Inventors: Kumi Oyama, Otsu-shi, Shiga (JP); Taira Omori, Nagoya-shi, Aichi (JP); Tomoko Takano, Otsu-shi, Shiga (JP)

(73) Assignee: Toray Industries, Inc., Tokyo (JP)

(21) Appl. No.: 18/265,507

(22) PCT Filed: Dec. 16, 2021
(86) PCT No.: PCT/JP2021/046545

§ 371 Date: Jun. 6, 2023

(30) Foreign Application Priority Data
Dec. 23, 2020 (JP) 2020-213308

(51) Int. Cl. A61K 8/02; A45D 44/00; A61Q 19/00; D04H 1/4258

(52) U.S. Cl. CPC A61K 8/0212; A45D 44/002; A61K 8/027; A61Q 19/00; D04H 1/4258

(57) ABSTRACT

There is provided a nonwoven fabric for skin care products excellent in adhesion and wiping properties, and soft touch to the skin. The nonwoven fabric includes synthetic fibers and cellulose fibers. When a maximum length of a cross section of the synthetic fibers is denoted by A and a maximum width thereof is denoted by B, flatness expressed by the formula (1) is 1.0 to 1.5:

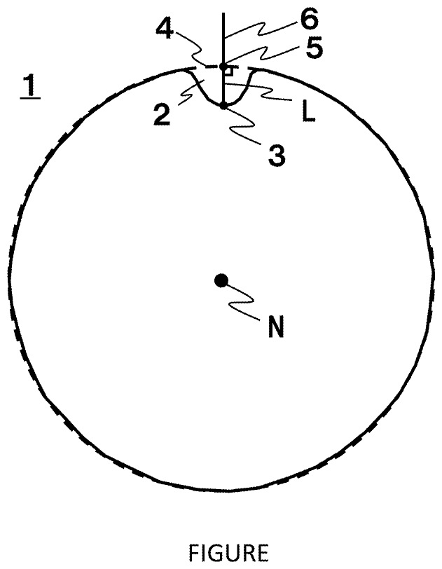

flatness=A/B (1)
the synthetic fibers have recesses, a depth of which is 1.0% or more and 10.0% or less relative to a single fiber diameter of the synthetic fibers, a single fiber diameter of the synthetic fibers is 1 μm or more and 20 μm or less, a content of the synthetic fibers is 30 to 70% by mass relative to the entire nonwoven fabric for skin care products, a content of the cellulose fibers is 70 to 30% by mass relative to the entire nonwoven fabric for skin care products, and an average single fiber diameter of all fibers constituting the nonwoven fabric for skin care products is 5 μm or more and 20 μm or less.

### CROSS REFERENCE TO RELATED APPLICATIONS

This is the U.S. National Phase application of PCT/JP2021/046545, filed Dec. 16, 2021, which claims priority to Japanese Patent Application No. 2020-213308, filed Dec. 23, 2020, the disclosures of each of these applications being incorporated herein by reference in their entireties for all purposes.

### FIELD OF THE INVENTION

The present invention relates to a nonwoven fabric for skin care products, a face mask, and a cleansing sheet.

### BACKGROUND OF THE INVENTION

In recent years, a demand for nonwoven fabrics has continued to expand as an application of skin care products that take advantage of liquid retention properties and feel. Then, sheet-like skin care products represented by face masks and cleansing sheets are used for various purposes such as penetration of a medicinal solution into skin and removal of cosmetics. The face masks can retain the medicinal solution on the surface of skin for a certain period of time and thus allows the medicinal solution to be sufficiently penetrated into the skin as compared with conventional skin care products applied directly to the skin. Furthermore, the cleansing sheet can provide a good cleansing effect due to both the effect of removing cosmetics by the cleansing agent and the effect of removing cosmetics by contact of the sheet with the skin. Due to the excellent performance as described above, various products have been proposed as sheet-like skin care products.

Recently, there has been proposed a nonwoven fabric for wiping use and cosmetic use for a human, using a nonwoven fabric including cellulosic fibers and modified cross section fibers having a cross-sectional shape whose form of a hole part is of an enclosed type (Patent Document 1). Then, Patent Document 1 discloses that the nonwoven fabric for wiping use and cosmetic use for a human described above has less irritation to the skin and also has good wiping properties.

Furthermore, there has been proposed a nonwoven fabric for wiping use and cosmetic use for a human using a nonwoven fabric including polyester fibers having a multi-lobed flattened cross-section and cellulosic fibers (Patent Document 2). Then, Patent Document 2 discloses that it has less irritation to the skin and also has good wiping properties.

Furthermore, there has been proposed a nonwoven fabric for a liquid-impregnated skin coating sheet using a nonwoven fabric including synthetic fibers having at least one recess or constriction in a fiber cross section and hydrophilic fibers (Patent Document 3). Then, Patent Document 3 discloses that the nonwoven fabric for the liquid-impregnated skin coating sheet described above exhibits a good adhesion.

### PATENT DOCUMENTS

Patent Document 1: Japanese Patent Laid-open Publication No. 2009-79320
Patent Document 2: WO 2014/132690 A
Patent Document 3: Japanese Patent Laid-open Publication No. 2019-170756

### SUMMARY OF THE INVENTION

According to findings of the present inventors, the nonwoven fabric disclosed in Patent Document 1 described above (hereinafter sometimes referred to as a known nonwoven fabric 1) includes cellulosic fibers and modified cross section fibers having a cross-sectional shape whose form of a hole part is of an enclosed type. Therefore, the nonwoven fabric for wiping use and cosmetic use for a human using the known nonwoven fabric 1 has less irritation to the skin and also has good wiping properties due to a cross-sectional shape of the modified cross section fiber described above. However, a smoothness of the surface of the known nonwoven fabric 1 tends to decrease, for example, in a case where a flatness of the modified cross section fiber included in the known nonwoven fabric 1 is large and a recess depth ratio to a single fiber diameter of the modified cross section fiber is large. Therefore, in a case where the known nonwoven fabric 1 is used as a nonwoven fabric for wiping use and cosmetic use for a human, there is a problem that a soft touch to the skin is insufficient.

Furthermore, there is a problem similar to that of the known nonwoven fabric 1 in the nonwoven fabric disclosed in Patent Document 2 (hereinafter sometimes referred to as a known nonwoven fabric 2). In other words, the smoothness of the surface of the known nonwoven fabric 2 tends to decrease, in a case where the flatness of a polyester fiber with a multi-lobed flattened cross section included in the known nonwoven fabric 2 is large and the depth of the recess is large relative to the single fiber diameter of the polyester fiber with a multi-lobed flattened cross-section. Therefore, in a case where the known nonwoven fabric 2 is used as a nonwoven fabric for wiping use and cosmetic use for a human, there is a problem that the soft touch to the skin is insufficient.

Furthermore, the nonwoven fabric disclosed in Patent Document 3 (hereinafter sometimes referred to as a known nonwoven fabric 3) includes the synthetic fibers having at least one recess or constriction in a fiber cross section and hydrophilic fibers. Therefore, the nonwoven fabric for the liquid-impregnated skin coating sheet using the known nonwoven fabric 3 exhibits a good adhesion. However, the smoothness of the surface of the known nonwoven fabric 3 tends to decrease, for example, in a case where the flatness of a synthetic fiber included in the known nonwoven fabric 3 is large and the depth of the recess is large relative to the single fiber diameter of the synthetic fiber. Therefore, in a case where the known nonwoven fabric 3 is used as a nonwoven fabric for a liquid-impregnated skin coating sheet, there is a problem that the soft touch to the skin is insufficient.

Therefore, in view of the circumstances described above, an object of the present invention is to provide a nonwoven fabric for skin care products, a face mask, and a cleansing sheet which are excellent in the adhesion to the skin and wiping properties against the cosmetics attached to the skin and excellent in soft touch to the skin. Note that the wiping properties above refer to the wiping properties of skin care products in a wet state, and the same applies hereinafter.

In order to solve the problems described above, an embodiment of the present invention includes the following composition. That is:

(1) A nonwoven fabric for skin care products containing synthetic fibers and cellulose fibers, wherein when a maximum length of a cross section of the synthetic fibers is denoted by A and a maximum width thereof is denoted by B, flatness expressed by the following formula (1) is 1.0 to 1.5:

flatness=A/B (1)

wherein the synthetic fibers have recesses, a depth of which is 1.0% or more and 10.0% or less relative to a single fiber diameter of the synthetic fibers, the single fiber diameter of the synthetic fibers is 1 μm or more and 20 μm or less, a content of the synthetic fibers is 30 to 70% by mass relative to the entire nonwoven fabric for skin care products, a content of the cellulose fibers is 70 to 30% by mass relative to the entire nonwoven fabric for skin care products, and an average single fiber diameter of all fibers constituting the nonwoven fabric for skin care products is 5 μm or more and 20 μm or less.

(2) The nonwoven fabric for skin care products according to (1), wherein the single fiber diameter of the synthetic fibers is 1 μm or more and 15 μm or less, and the average single fiber diameter of all fibers constituting the nonwoven fabric for skin care products is 5 μm or more and 15 μm or less.

(3) The nonwoven fabric for skin care products according to (1) or (2), wherein a tensile strength of the synthetic fibers measured in accordance with JIS L1015 2010 8.7.1 is 2.5 to 3.2 cN/dtex.

(4) The nonwoven fabric for skin care products according to any one of (1) to (3), the nonwoven fabric having a laminated nonwoven fabric structure including a first layer and a second layer,

wherein the first layer constitutes one surface of the nonwoven fabric for skin care products, the first layer contains the synthetic fibers in an amount of 50% by mass or more relative to the entire first layer, and a variation in the single fiber diameter of the synthetic fibers in the first layer is 1 to 10%,

the average single fiber diameter of fibers constituting the second layer is 5 to 15 μm, and a variation in the single fiber diameter of fibers constituting the second layer is 11 to 35%, and

fibers constituting the first layer and fibers constituting the second layer are entangled.

(5) A face mask having the nonwoven fabric for skin care products according to any one of (1) to (4).

(6) A cleansing sheet having the nonwoven fabric for skin care products according to any one of (1) to (4).

According to the present invention, it is possible to provide a nonwoven fabric for skin care products, a face mask, and a cleansing sheet which are excellent in an adhesion to the skin and wiping properties against the cosmetics attached to the skin, and excellent in soft touch to the skin.

### BRIEF DESCRIPTION OF THE DRAWINGS

The FIGURE is a conceptual diagram of a cross-sectional shape of synthetic fibers contained in one embodiment of a nonwoven fabric for skin care products of the present invention.

### DETAILED DESCRIPTION OF EMBODIMENTS OF THE INVENTION

Hereinafter, embodiments of the present invention will be described in detail.

The nonwoven fabric for skin care products according to embodiments of the present invention has the following composition. In other words, a nonwoven fabric for skin care products containing synthetic fibers and cellulose fibers, wherein when a maximum length of a cross section of the synthetic fibers is denoted by A and a maximum width thereof is denoted by B, flatness expressed by the following formula (1) is 1.0 to 1.5:

flatness=A/B (1)

wherein the synthetic fibers have recesses, a depth of which is 1.0% or more and 10.0% or less relative to a single fiber diameter of the synthetic fibers, the single fiber diameter of the synthetic fibers is 1 μm or more and 20 μm or less, a content of the synthetic fibers is 30 to 70% by mass relative to the entire nonwoven fabric for skin care products, a content of the cellulose fibers is 70 to 30% by mass relative to the entire nonwoven fabric for skin care products, and an average single fiber diameter of all fibers constituting the nonwoven fabric for skin care products is 5 μm or more and 20 μm or less.

The nonwoven fabric for skin care products according to embodiments of the present invention includes the synthetic fibers. To start with, such synthetic fibers will be described.

Materials of the synthetic fibers to be used in the present invention are not particularly limited, but include polyester resins, polyamide resins, acrylic resins, polyolefin resins, and the like. Among these materials, the acrylic resins are preferable because they are hydrophilic and make the nonwoven fabric for skin care products more flexible due to swelling of the synthetic fibers in a wet state, and consequently make the nonwoven fabric for skin care products excellent in the adhesion to the skin, wiping properties, and soft touch to the skin. Further, these materials may be copolymerized with other components, or may contain additives such as stabilizers.

Furthermore, a synthetic fiber used in the nonwoven fabric for skin care products according to embodiments of the present invention has flatness of 1.0 to 1.5 expressed by the formula (1) when the maximum length of a cross-section of the synthetic fiber is denoted by A and the maximum width thereof is denoted by B, and the synthetic fiber has a recess, the depth of which is 1.0% or more and 10.0% or less relative to the single fiber diameter of the synthetic fiber. Hereinafter, note that the recess depth ratio in the synthetic fiber to the single fiber diameter of the synthetic fiber may be simply referred to as a recess depth ratio in the synthetic fiber.

By setting the flatness to 1.0 to 1.5, deterioration of yarn manufacturing properties of the synthetic fibers can be suppressed. Furthermore, in skin care products using the nonwoven fabric for skin care products, a uniformity of an orientation of the synthetic fibers present on the surface of the skin care products is increased. Then, as a result, the soft touch to the skin of the skin care products is excellent. For the above reason, the flatness is more preferably 1.0 to 1.4, and still more preferably 1.0 to 1.3. Furthermore, the cross-sectional shape of the synthetic fiber is not particularly limited, but for the above reason, it is preferably not a cross-sectional shape having a sharp end portion, and more preferably a substantially circular shape. Examples of the cross-sectional shape having the sharp end portion include a polygon such as a quadrangle or a hexagon.

Here, the maximum length A of the cross section of the synthetic fiber refers to a length of the longest line segment measured when a length of a line segment from a certain point to another point on an outer periphery of the cross-sectional shape is measured in a cross section perpendicular to a fiber axis direction of the synthetic fibers. Furthermore, the maximum width B refers to the maximum length among the line segments connecting two intersections where a straight line perpendicular to the line segment (a long axis) of the maximum length A above intersects an outer periphery of the fiber cross section. Note that, the flatness of the synthetic fiber can be measured by photographing an image with a scanning electron microscope (SEM).

Furthermore, by setting the recess depth ratio in the synthetic fiber to 1.0% or more, in the skin care products using the nonwoven fabric for skin care products according to embodiments of the present invention, the synthetic fibers present on the surface of the skin care products follow fine irregularities present in the skin, a contact area between the skin care products and the skin increases, and a coefficient of friction between the skin care products and the skin increases. Then, as a result, the adhesion and wiping properties of the skin care products become excellent. On the other hand, by setting the recess depth ratio in the synthetic fiber to 10.0% or less, the smoothness of the surface of the synthetic fibers is improved. In this way, irritation to the skin and deterioration of tactile sensation can be suppressed during using the skin care products that uses the nonwoven fabric for skin care products according to embodiments of the present invention, and thus the soft touch to the skin of the skin care products is excellent. For the above reason, the recess depth ratio in the synthetic fiber is more preferably 8.0% or less, and still more preferably 6.0% or less.

Here, the depth of the recesses in the synthetic fibers will be described in detail with reference to the figure. The FIGURE is a conceptual diagram of a cross-sectional shape of a synthetic fiber contained in one embodiment of the nonwoven fabric for skin care products of the present invention. As shown in the FIGURE, a cross section 1 of the synthetic fiber has one recess 2. The depth of the recess in the synthetic fiber is a length L of a line segment connecting a deepest point 3 and a contact point 5 between a circumscribed circle 4 and a perpendicular line 6 drawn in a direction substantially opposite to a center point N of the cross section 1 among the perpendicular lines (the lines perpendicular to a tangent line of the circumscribed circle 4) drawn from the deepest point 3 of the recess 2 shown in the FIGURE to the circumscribed circle 4 of the cross section 1. Furthermore, the depth of the recess in the synthetic fiber can be calculated by photographing a cross-sectional photograph of the nonwoven fabric for skin care products with a scanning electron microscope (SEM) and measuring the length of the depth L of the recess in the synthetic fiber in the same photograph.

Note that, the recess in the synthetic fiber to be used in an embodiment the present invention can be confirmed as a linear form in a direction substantially parallel to a longitudinal direction of the fiber even on a side surface of the synthetic fiber, and the recess may be in a continuous linear form or a discontinuous linear form, that is, it may be in a so-called linear form regardless of its length.

The synthetic fiber to be used in an embodiment of the present invention can be produced by spinning in a normal manner suitable for a type of the polymer to be used, but at that time, the shape of a discharge hole and spinning conditions are appropriately adjusted and controlled so as to form a cross-sectional shape defined in the present invention. Note that for example, the fine irregularities are formed on a fiber surface by dropping inorganic fine particles from the surface of polyethylene terephthalate fibers by alkali reduction, but in this embodiment, it is not a preferred form because some of the inorganic fine particles above may remain on the fiber surface without falling off, and then, as a result, it may cause the irritation to the skin and the deterioration in tactile sensation when the skin care products using the nonwoven fabric for skin care products of the present invention are used. Therefore, the content of inorganic fine particles in the synthetic fiber is preferably set to 0% by mass, but even when it is contained, it is preferably limited to an extent in which the irritation to the skin and deterioration of tactile sensation are not caused, for example, 0.1% by mass or less.

Since the recess depth ratio in the synthetic fiber to be used in embodiments of the present invention is 1.0% or more and 10.0% or less, in the skin care products using the nonwoven fabric for skin care products containing the synthetic fiber, the synthetic fiber follows the fine irregularities present in the skin, the contact area between the skin care products and the skin increases, and the coefficient of friction between the skin care products and the skin increases. Then, as a result, it is presumed that the adhesion and wiping properties of the skin care products are improved. As a method for evaluating the coefficient of friction between the skin care products and the skin, it can be evaluated by an average coefficient of dynamic friction (MIU) measured using a roughness/friction tester (KES-SESRU manufactured by Kato Tech Co., Ltd.). From a viewpoint that the skin care products have more excellent adhesion to the skin surface and the skin care products have more excellent wiping properties, the average coefficient of dynamic friction (MIU) of the nonwoven fabric for skin care products is preferably 0.058 or more, more preferably 0.060 or more, still more preferably 0.062 or more, and particularly preferably 0.070 or more.

Furthermore, since the synthetic fiber to be used in embodiments of the present invention has the flatness of 1.0 to 1.5, the uniformity of the orientation of the synthetic fibers present on the surface of the skin care products is improved for the skin care products using the nonwoven fabric for skin care products containing the synthetic fibers. Further, since the recess depth ratio in the synthetic fiber is 1.0% or more and 10.0% or less, the smoothness of the synthetic fiber surface is improved, and the irritation to the skin and deterioration of tactile sensation can be suppressed when the skin care products using the nonwoven fabric for skin care products containing the synthetic fiber are used. Then, as a result, it is presumed that the soft touch to the skin of the skin care products is improved because the irritation to the skin and deterioration of tactile sensation can be suppressed in the skin care products. On the other hand, the recesses in the synthetic fibers follow the fine irregularities present in the skin, the contact area between the skin care products and the skin increases, and the coefficient of friction between the skin care products and the skin increases. Then, as a result, it is presumed that the skin care products also have excellent adhesion and wiping properties. Note that, as a method for evaluating surface characteristics of the skin care products, which has the soft touch to the skin but also has excellent adhesion and wiping properties, values of the average coefficient of dynamic friction (MIU), a variation in the average coefficient of dynamic friction (MMD), and a standard deviation of the average surface roughness (SMD) are measured using a roughness/friction tester (KES-SESRU manufactured by Kato Tech Co., Ltd.), and an evaluation can be performed according to the surface characteristics calculated by the following formula (2). From a viewpoint that the skin care products have a more excellent soft touch to the skin, the surface characteristics of the nonwoven fabric for skin care products are preferably 2.3 or more, more preferably 2.5 or more, still more preferably 3.0 or more, and particularly preferably 3.5 or more.

Surface characteristics=MIU/(MMD×SMD) (2)

The synthetic fiber to be used in embodiments of the present invention has a single fiber diameter of 1 μm or more and 20 μm or less. By setting the single fiber diameter of the synthetic fiber to 1 μm or more, productivity of the nonwoven fabric for skin care products can be improved. Then, handling properties of the skin care products can be further improved. For the above reason, a lower limit of the single fiber diameter of the synthetic fiber is more preferably 5 μm or more, and still more preferably 8 μm or more. On the other hand, by setting the single fiber diameter to 20 μm or less, the synthetic fiber is prevented from becoming rigid, and the synthetic fibers present on the surface of the skin care products highly follow the fine irregularities of the skin in the skin care products using the nonwoven fabric for skin care products according to embodiments of the present invention. Then, the adhesion and wiping properties of the skin care products are further improved. For the above reason, an upper limit of the single fiber diameter of the synthetic fiber is more preferably 15 μm or less, and still more preferably 10 μm or less. Here, following the skin of the skin care products refers to, for example, a performance that the face mask is attached along the irregularities present on the user's face (the region from a tip of the user's nose to the cheek, and the like) when it is used. Here, the single fiber diameter of the synthetic fiber is a diameter of a perfect circle having the same area as an area of the cross section of the synthetic fiber measured from the cross-sectional photograph of the nonwoven fabric for skin care products.

Furthermore, the variation in the single fiber diameter of the synthetic fibers is not particularly limited, but is preferably 20% or less. When the variation in the single fiber diameter of the synthetic fibers is 20% or less, the single fiber diameter of the synthetic fibers becomes uniform. As a result, the smoothness of the synthetic fibers present on the surface of the skin care products using the nonwoven fabric for skin care products according to embodiments of the present invention is further improved. For the above reason, the upper limit of the variation in the single fiber diameter of the synthetic fibers is more preferably 15% or less, and still more preferably 10% or less. Then, as a result, the soft touch to the skin of the skin care products is excellent. Note that, the variation in the single fiber diameter of the synthetic fibers can be measured by photographing an image with a scanning electron microscope (SEM).

The tensile strength of the synthetic fiber to be used in the present invention is preferably 2.5 to 3.2 cN/dtex. By containing synthetic fibers having a tensile strength of 2.5 to 3.2 cN/dtex and moderate flexibility, the adhesion and wiping properties of the skin care products using the nonwoven fabric for skin care products according to embodiments of the present invention are further improved. Note that, the tensile strength of the synthetic fibers is a tensile strength measured in accordance with JIS L1015 2010 8.7.1.

The content of the synthetic fibers in embodiments of the present invention is 30 to 70% by mass relative to the entire nonwoven fabric for skin care products. Since the synthetic fiber has a recess at a specific ratio relative to the single fiber diameter of the synthetic fiber, by setting the content of the synthetic fibers to 30% by mass or more, in the skin care products using the nonwoven fabric for skin care products according to embodiments of the present invention, the synthetic fibers present on the surface of the skin care products follow the fine irregularities present in the skin, the contact area between the skin care products and the skin increases, and the coefficient of friction between the skin care products and the skin increases. Then, as a result, the adhesion and wiping properties of the skin care products become excellent. Further, since the synthetic fiber has a recess at a specific ratio, the smoothness of the fiber surface is improved, so that the soft touch to the skin of the skin care products becomes excellent. For the above reason, the lower limit of the content of the synthetic fibers is more preferably 40% by mass or more, and still more preferably 50% by mass or more. On the other hand, by setting the content of the synthetic fibers to 70% by mass or less, the content of the cellulose fibers is increased, and a balance between the adhesion and wiping properties, as well as the soft touch to the skin and the liquid retention properties of the skin care products using the nonwoven fabric for skin care products is excellent.

Furthermore, as the cellulose fiber of the present invention, a plant-based natural fiber such as pulp and cotton, a regenerated fiber such as rayon and cupra, and a semi-synthetic fiber such as acetate and triacetate can be used. Among these, the rayon is preferable because it can further improve the adhesion and wiping properties of the skin care products.

On the other hand, the content of the cellulose fibers in embodiments of the present invention is 70 to 30% by mass. By setting the content of the cellulose fibers to 70 to 30% by mass, the liquid retention properties of the skin care products using the nonwoven fabric for skin care products is excellent. For the above reason, the lower limit of the content of the cellulose fibers is more preferably 40% by mass or more, and still more preferably 50% by mass or more.

The average single fiber diameter of all fibers constituting the nonwoven fabric for skin care products according to embodiments of the present invention (hereinafter sometimes referred to as an average single fiber diameter) is 5 μm or more and 20 μm or less. By setting the average single fiber diameter to 5 μm or more, a strength of the nonwoven fabric for skin care products can be improved. Then, as a result, the productivity and the handling properties during use of the nonwoven fabric for skin care products can be improved. On the other hand, by setting the average single fiber diameter to 20 μm or less, the nonwoven fabric for skin care products according to embodiments of the present invention becomes excellent in flexibility. As a result, the skin care products using a nonwoven fabric for skin care products can be flexibly bent along fine irregularities of the user's skin, and the skin following properties of the skin care products is excellent. Then, as a result, the adhesion and wiping properties of the skin care products are further improved. For the above reason, the upper limit of the single fiber diameter of the synthetic fiber is more preferably 15 μm or less, and still more preferably 13 μm or less.

The nonwoven fabric for skin care products in embodiment of the present invention may have a homogeneous nonwoven fabric structure as a whole, but may have the so-called laminated nonwoven fabric structure having a plurality of layers with different compositions in a thickness direction.

Among these, it is preferable to use a nonwoven fabric for skin care products having a laminated nonwoven fabric structure including a first layer and a second layer, wherein the first layer constitutes one surface of the nonwoven fabric for skin care products, the first layer contains the synthetic fibers in an amount of 50% by mass or more relative to the entire first layer, and a variation in the single fiber diameter of the synthetic fibers in the first layer is 1 to 10%, the average single fiber diameter of fibers constituting the second layer is 5 to 15 μm, and a variation in the single fiber diameter of fibers constituting the second layer is 11 to 35%, and fibers constituting the first layer and fibers constituting the second layer are entangled.

In the nonwoven fabric for skin care products according to embodiments of the present invention, the smoothness of the synthetic fibers present on the surface of the skin care products using the nonwoven fabric for skin care products according to embodiments of the present invention is further improved, since the first layer contains the synthetic fibers having a small variation in the single fiber diameter in an amount of 50% by mass or more relative to the entire first layer. Furthermore, in the skin care products using the nonwoven fabric for skin care products according to embodiments of the present invention, the synthetic fibers present on the surface of the skin care products follow the fine irregularities present in the skin, the contact area between the skin care products and the skin increases, and the coefficient of friction between the skin care products and the skin increases. Then, as a result, the adhesion and wiping properties of the skin care products become excellent. Further, since the synthetic fiber has a recess at a specific ratio and the smoothness of the fiber surface is improved, the soft touch to the skin of the skin care products becomes excellent. For the above reason, the upper limit of the variation in the single fiber diameter of the synthetic fibers is more preferably 9% or less, and still more preferably 8% or less. Furthermore, the lower limit of the content of the synthetic fibers is more preferably 70% or more, and still more preferably 100%.

The content of the synthetic fibers in the first layer and the second layer, the average single fiber diameter of each fiber, the variation in the single fiber diameter, and the like are determined by a method to be described later.

Further, in the nonwoven fabric for skin care products according to embodiments of the present invention, since the average single fiber diameter and the variation in the single fiber diameter of the fibers constituting the second layer are within a specific range, a suitable gap can be maintained between the fibers constituting the nonwoven fabric for skin care products. As a result, the nonwoven fabric for skin care products becomes flexible. Then, as a result, the adhesion and wiping properties of the skin care products using the flexible nonwoven fabric for skin care products are improved. For the above reason, the upper limit of the average single fiber diameter of the fibers constituting the second layer is more preferably 13 μm or less, and still more preferably 12 μm or less. Furthermore, a preferred range of the variation in the single fiber diameter of the fibers constituting the second layer is as described above, but the upper limit thereof is more preferably 30% or less, and still more preferably 20% or less.

Then, the first layer and the second layer are preferably integrated by being entangled.

From a viewpoint that skin care products using the nonwoven fabric for skin care products of the present invention have excellent adhesion and wiping properties, and soft touch to the skin, specific aspects of the nonwoven fabric for skin care products having the laminated nonwoven fabric structure include a two-layer laminated nonwoven fabric structure (first layer/second layer) in which the first layer is disposed on one surface of the second layer, a three-layer laminated nonwoven fabric structure (first layer/second layer/first layer) in which the first layer is disposed on both surfaces of the second layer, and the like.

A basis weight of the nonwoven fabric for skin care products of the present invention is preferably 80 g/m² or less, more preferably 65 g/m² or less, and still more preferably 50 g/m² or less. By setting the basis weight to 80 g/m² or less, the flexibility of the nonwoven fabric for skin care products of the present invention is improved, and as a result, the adhesion and wiping properties of the skin care products using the nonwoven fabric for skin care products are improved. Note that, the basis weight of the present invention can be measured according to JIS L1913: 2010 6.2.

The nonwoven fabric for skin care products of the present invention may contain synthetic fibers having a single fiber diameter of 20 μm or more, or fibers other than the synthetic fibers and cellulose fibers, as long as the effect is not impaired. Examples of fibers other than the synthetic fibers and cellulose fibers include animal-based natural fibers such as silk and wool.

Furthermore, the nonwoven fabric for skin care products of the present invention preferably has a strength of 5.0 N/25 mm or less at 10% elongation when wet measured in accordance with JIS L1913: 2010.6.3.2. Here, the strength at 10% extension when wet described above is an average value of, the strength at 10% extension when wet in a traveling direction of the nonwoven fabric for skin care products (hereinafter sometimes referred to as an MD direction) in the manufacturing process of the nonwoven fabric for skin care products, and the strength at 10% extension when wet in a direction substantially perpendicular to a traveling direction of the nonwoven fabric for skin care products (hereinafter sometimes referred to as a CD direction) in the manufacturing process of the nonwoven fabric for skin care products.

The nonwoven fabric for skin care products according to embodiments of the present invention has a strength at 10% elongation when wet described above within a range of 5.0 N/25 mm or less, so that the nonwoven fabric for skin care products is flexible. Then, as a result, the adhesion and wiping properties of the skin care products using the flexible nonwoven fabric for skin care products are improved. As described above, the adhesion and wiping properties become excellent. To make the nonwoven fabric for skin care products become more flexible and the adhesion and wiping properties of the skin care products using the nonwoven fabric for skin care products become more excellent, the strength at 10% elongation when wet of the nonwoven fabric for skin care products is more preferably 4.0 N/25 mm or less, still more preferably 3.0 N/25 mm or less, and particularly preferably 2.0 N/25 mm or less.

Furthermore, since the nonwoven fabric for skin care products according to embodiments of the present invention contains the cellulose fibers, in the skin care products using the nonwoven fabric for skin care products according to embodiments of the present invention, the cellulose fibers take in a medicinal solution into the fibers. As a result, the medicinal solution can be prevented from volatilizing in the air, and the liquid retention properties of the skin care products are improved. As a result, it is presumed that this contributes to the improvement of wiping properties of the skin care products. As a method for evaluating the liquid retention properties, it can be evaluated by a water absorption ratio calculated using a value of the mass of the nonwoven fabric for skin care products under standard conditions and a value of the mass of the nonwoven fabric for skin care products after impregnating the nonwoven fabric for skin care products with distilled water for 10 minutes according to JIS L1906: 2010 3. Water Absorption. From a viewpoint that the wiping properties of the skin care products are more excellent, the water absorption ratio of the nonwoven fabric for skin care products is preferably 7.0 or more, more preferably 8.0 or more, and still more preferably 9.0 or more.

Next, the nonwoven fabric for skin care products of the present invention is preferably a dry nonwoven fabric. By using the dry nonwoven fabric, a thickness of the nonwoven fabric can be made larger than that of a wet nonwoven fabric, softness of the nonwoven fabric is improved, and as a result, the adhesion and wiping properties of the skin care products using the nonwoven fabric for skin care products can be improved. Further, the nonwoven fabric for skin care products of the present invention is preferably a spun-laced nonwoven fabric among the dry nonwoven fabrics. The spun-laced nonwoven fabric can be obtained by a method of entangling constituent fibers with a high-pressure water flow, but in this method, compared to a method of entangling the constituent fibers by needle punching, thread breakage of the constituent fibers during entanglement is small, and the smoothness of the surface of the nonwoven fabric can be excellent. As a result, a nonwoven fabric for skin care products having an excellent soft touch to the skin can be obtained. Further, in this method, by injecting the high-pressure water flow for a plurality of times from front and back surfaces of the nonwoven fabric for skin care products according to embodiments of the present invention, since water absorption of the cellulose fibers contained in the nonwoven fabric for skin care products is superior to that of the synthetic fibers contained in the nonwoven fabric for skin care products, entanglement of the cellulose fibers is improved compared to that of the synthetic fibers, and the cellulose fibers are preferentially disposed inside the nonwoven fabric for skin care products. Then, as a result, the synthetic fibers are preferentially disposed on the front and back surfaces of the nonwoven fabric for skin care products, which is preferable since the adhesion and wiping properties, and soft touch to the skin of the skin care products using the nonwoven fabric for skin care products are excellent. Number of times of injecting the high-pressure water flow is preferably 5 or more in total, and a water pressure is preferably 1 to 10 MPa during a first to a third injection, and is preferably 10 to 15 MPa after a fourth injection.

Furthermore, in a case where the nonwoven fabric for skin care products according to embodiments of the present invention has the laminated nonwoven fabric structure including the first layer and the second layer, the first layer and the second layer may be individually entangled by the high-pressure water flow, and then the first layer and the second layer may be laminated and integrated by the high-pressure water flow to form a laminated nonwoven fabric structure, or a web of the first layer and a web of the second layer after a carding step (before the entanglement) may be laminated, and entangled and integrated by the high-pressure water flow to form the laminated nonwoven fabric structure. Further, in a case where the web of the first layer and the web of the second layer are laminated, and entangled and integrated by the high-pressure water flow, the high-pressure water flow may be injected for 1 to 5 times each from the front and back surfaces of the laminated fiber web, and may be injected for 5 times or more in total. The water pressure is preferably 1 to 10 MPa during the first to the third injections, and is preferably 10 to 15 MPa after the fourth injection.

Note that, in a case where the nonwoven fabric for skin care products according to embodiments of the present invention has the laminated nonwoven fabric structure, since the fibers constituting the first layer and the fibers constituting the second layer are entangled with each other, a boundary thereof may gradually change and may not be clear.

Then, the skin care products can be obtained by immersing the nonwoven fabric for skin care products according to embodiments of the present invention in a medicinal solution such as a lotion, a serum, or a cleansing agent. Here, examples of the skin care products include face masks, skin care sheets, eye masks, and cleansing sheets (including cleansing sheets for point makeup in addition to normal cleansing sheets), and the like.

### EXAMPLES

The measuring method used in present Examples will be described below.

(1) Single Fiber Diameter, Recess Depth Ratio and Flatness of Fiber

The nonwoven fabric for skin care products was cut perpendicularly to the surface of the nonwoven fabric for skin care products, a thin section was cut out from the nonwoven fabric for skin care products, and a Pt—Pd (platinum-palladium) alloy was vacuum-deposited on the cross section of the thin section to obtain a deposited body. Next, a cross-sectional portion of the nonwoven fabric included in the deposited body was observed with a scanning electron microscope (SEM) (SU3800 type manufactured by Hitachi High-Tech Co., Ltd.), 10 points were randomly extracted from this observation range, and the cross-sectional photograph was taken at a magnification of 1,000 times. Next, in the same photograph, 10 synthetic fibers having recesses were randomly extracted, and for each of a total of 100 synthetic fibers, the ratio of the single fiber diameter to the depth of the recess as well as the flatness in the cross section of the fiber were measured to determine the depth of the recess to the single fiber diameter and the flatness.

A fiber having the depth of recess in 1.0% or more and 10.0% or less relative to a single fiber diameter, a flatness of 1.0 to 1.4, and a single fiber diameter of 1 μm or more and 20 μm or less were used as the synthetic fiber in embodiments of the present invention. Note that, a specific measurement method is as described in detail below.

1) Single Fiber Diameter of Synthetic Fibers

The single fiber diameter of each of a total of 100 synthetic fibers extracted in the above (1) was measured, and an average value of the total of 100 measured values obtained was taken as the single fiber diameter of the synthetic fibers. Note that, in a case where the synthetic fibers had a modified cross-sectional shape, the cross-sectional area of the fiber was measured from the cross-sectional photograph, and the cross-sectional area was converted into a perfect circle diameter to obtain the single fiber diameter of the synthetic fibers.

2) Flatness of Fibers

For a total of 100 synthetic fibers extracted in the above (1), the maximum length A and the maximum width B in the cross section of the fibers were measured, the flatness was obtained by the following formula (1), and an average value of the total of 100 calculated values obtained was taken as the flatness of the synthetic fibers.

flatness=A/B (1)

3) Depth of Recess in Synthetic Fiber and Recess Depth Ratio in Synthetic Fiber

For a total of 100 synthetic fibers extracted in the above (1), the cross-sectional photograph was taken at a magnification of 5,000 times. Next, in the same photograph, the depth of the recesses (the depth of the recesses corresponding to the length of a line segment L exemplified in the FIGURE) in the cross section of the fibers was measured, and an average value of the total of 100 measured values obtained was taken as the depth of the recesses in the cross section of the synthetic fibers. Note that the depth of the recess was as described in detail above with reference to the FIGURE, and in a case where there is a plurality of recesses in the cross section of a fiber, the depth of the recess in the synthetic fiber was defined as the maximum length.

Next, from the single fiber diameter and the depth of the recess in the same fiber measured as described above, the recess depth ratio to the single fiber diameter of the synthetic fiber was determined by the following formula (3), and an average value of the 100 calculated values obtained was taken as the recess depth ratio to the single fiber diameter of the synthetic fiber.

Recess depth ratio to single fiber diameter of synthetic fiber C=depth of recess in synthetic fiber C (length of line segment L)/single fiber diameter of synthetic fiber C×100(%) (3)

(2) Variation in Single Fiber Diameter of Synthetic Fibers

The variation in the single fiber diameter of the synthetic fibers contained in the nonwoven fabric for skin care products was measured as follows. Specifically, the variation in the single fiber diameter of the synthetic fibers was calculated by the following formula (4) using the value of the single fiber diameter of each of the synthetic fibers measured in the above (1).

Variation in single fiber diameter of synthetic fibers (single fiber diameter CV % of synthetic fibers)=(standard deviation of single fiber diameter of synthetic fibers/average value of single fiber diameter of synthetic fibers)×100(%) (4)

(3) Identification of Materials and Contents of Synthetic Fibers, Cellulose Fibers, and Other Fibers Contained in Nonwoven Fabric for Skin Care Products

The synthetic fibers specified in the above (1), the cellulose fibers, and other fibers contained in the nonwoven fabric for skin care products were identified, and the materials were identified according to JIS L1030-1: 2012 “Testing methods for quantitative analysis of fiber mixture ratio of textiles—Part 1: Testing methods for fiber identification” and JIS L1030-2: 2012 “Testing methods for quantitative analysis of fiber mixture ratio of textiles—Part 2: Testing methods for quantitative analysis of fiber mixtures”. Furthermore, the mixture ratio based on corrected mass (a mass ratio of each fiber under standard conditions) of the synthetic fibers, the cellulose fibers, and the other fibers contained in the nonwoven fabric for skin care products whose materials were specified above was measured, and this was taken as the content (mass %) of the synthetic fibers, the cellulose fibers, and the other fibers contained in the nonwoven fabric for skin care products.

(4) Identification of Laminated Nonwoven Fabric Structure

A 50 mm×50 mm test piece was obtained from a sample using a steel ruler and a razor blade. Next, an attempt has been made that, two tweezers were prepared, one of the two tweezers was used to grasp a vicinity of the surface of one surface of the test piece, the other tweezer of the two tweezers was used to grasp a vicinity of the surface of the other surface of the test piece, and the two tweezers were pulled in a direction substantially perpendicular to both directions of the test piece to make the test piece into two sheet-like separation test pieces. In a case where two separation test pieces were obtained, the same operation as the above operation was repeated for each separation test piece. The above operation was repeated until no separation test piece was obtained according to the above operation. Here, the sample from which the separation test piece was obtained was identified as a laminated nonwoven fabric. Note that, the separation test piece corresponds to each layer of the laminated nonwoven fabric.

(5) Identification of First Layer and Second Layer

Each of the plurality of separation test pieces obtained by performing the above operation of (4) was identified as the first layer and the second layer in the following method.

1) Identification of First Layer

For each of the plurality of separation test pieces obtained by performing the above operation (4), in a portion of the separation test piece that is not gripped with the tweezers, the single fiber diameter, the recess depth ratio to the single fiber diameter, and the flatness of the synthetic fibers having recesses were obtained by the same operation as in the above (1), and it was confirmed that the synthetic fibers had the depth of the recesses in 1.0% or more and 10.0% or less relative to the single fiber diameter, the flatness was 1.0 to 1.4, and the single fiber diameter was within the range of 1 μm or more and 20 μm or less. Next, the variation in the single fiber diameter of the synthetic fibers was determined by the same operation as in the above (2), the content of the synthetic fibers was calculated by the same operation as in the above (3), and a separation test piece, was defined as the first layer, in which the variation in the single fiber diameter of the synthetic fibers was 1 to 10% and the content of the synthetic fibers was 50% by mass or more.

2) Identification of Second Layer

For each of the plurality of separation test pieces obtained by performing the above operation (4), the cross-sectional photograph was taken at a magnification of 1,000 times by the same operation as in the above (1) at a portion of the separation test piece that is not gripped by the tweezers. Next, in the same photograph, 10 fibers were randomly extracted, the single fiber diameter was measured for a total of 100 fibers, and an average value of the 100 measured values obtained was taken as the average single fiber diameter of the fibers constituting the separation test piece. Next, the variation in the single fiber diameter of the fibers constituting the separation test piece was measured by the same operation as in the above (2), and the separation test piece in which the average single fiber diameter was 5 to 15 μm and the variation in the single fiber diameter of the fibers was 11 to 35% was defined as the second layer.

Furthermore, the sample from which no separation test piece was obtained in the above (4) was also identified as the first layer and the second layer by the following method.

3) Identification of First Layer and Second Layer in Sample from which No Separation Test Piece was Obtained

For the sample from which no separation test piece was obtained in the above (4), the cross-sectional photograph was taken at a magnification of 50 times by the same operation as in the above (1). Next, in a case where the boundary of each layer structure can be clearly identified visually in the same photograph, the first layer and the second layer were identified by the same operation as in (5)-1) and (5)-2) above for each layer identified.

Furthermore, in a case where the boundary of the layer structure cannot be clearly identified visually in the cross-sectional photograph, the cross-sectional photograph at a magnification of 1,000 times was taken for an observation range extracted at 20 points in a direction substantially perpendicular to the surface of the nonwoven fabric for skin care products from one surface layer toward the other surface layer of the nonwoven fabric for skin care products by the same operation as in the above (1). Subsequently, in the same photograph, the synthetic fibers having a variation in the single fiber diameter of 1 to 10% were identified by the same operations as in the above (1) and (2). Next, by the same operation as in the above (3), the materials of the synthetic fibers, the cellulose fibers, and other fibers were identified, the content of the synthetic fibers was calculated from the number and a specific gravity of each fiber observed in the same photograph, and all cross-sectional photographs with a synthetic fiber content of 50% by mass or more were identified. Next, a region including the observation range of these cross-sectional photographs and substantially parallel to the surface of the nonwoven fabric for skin care products was defined as the first layer. On the other hand, for the region where the content of the synthetic fibers was less than 50% by mass, the second layer was identified by performing the same operation as in the above (5)-2).

(6) Average Single Fiber Diameter of all Fibers Constituting Nonwoven Fabric for Skin Care Products

Ten fibers were randomly extracted from each of the 10 cross-sectional photographs of the nonwoven fabric for skin care products with a magnification of 1,000 times taken in the above (1), and the cross-sectional area of a total of 100 fibers was measured, the single fiber diameter of the fibers was measured by converting the cross-sectional area into a perfect circle diameter, and the average value of the single fiber diameters of the total of 100 fibers was calculated as the average single fiber diameter of all fibers constituting the nonwoven fabric for skin care products. Note that, for a sample having the laminated structure, the cross-sectional photograph shall be taken so that the object to be measured is uniformly extracted in the thickness direction.

(7) Tensile Strength of Synthetic Fibers

The measurement was performed according to JIS L1015 2010 8.7.1. Specifically, 50 synthetic fibers were collected from the nonwoven fabric for skin care products. Next, section lines were made on a piece of paper with a smooth and glossy surface (a space distance of 20 mm), both ends of each of the 50 synthetic fibers were pasted and fixed with an adhesive in a state of being loosely stretched within the section one by one, and then one sample was obtained for each section. Next, one sample was attached to a grip of a constant speed elongation tensile tester, and a paper piece was cut near an upper grip and measured. The grip interval was set to 20 mm, a load was applied at a tensile speed of 20 ram/min until the sample was cut, the strength at that time was measured, and this measurement was performed on 50 synthetic fibers. An average value of 50 measurement results was calculated, and the tensile strength of the synthetic fiber was calculated. Additionally, in a case where the test could not be performed with a grip interval of 20 mm since the fibers were short, the grip interval was set to 10 mm, a load was applied at a tensile speed of 10 ram/min until the sample was cut, and the strength at that time was measured.

(8) Basis Weight

The measurement was performed according to JIS L1913:2010 6.2. Specifically, three test pieces of 50 mm×50 mm were collected from the sample of the nonwoven fabric for skin care products using the steel ruler and the razor blade. A mass of the test piece under standard conditions was measured, the mass per unit area was obtained by the following formula, and the average value was calculated.

ms=m/S

Here, ms: mass per unit area (g/m²),
m: average weight of test piece (g), and
S: area of test piece (m²).

(9) Thickness

The measurement was performed according to JIS L1913: 2010 6.1.2 A method.

Five test pieces of 50 mm×50 mm were collected from the sample of the nonwoven fabric for skin care products using the steel ruler and the razor blade. Using a thickness measuring instrument (a constant pressure thickness measuring instrument manufactured by TECLOCK, model PG11J), the thickness was measured by applying a pressure of 0.36 kPa to the test piece under standard conditions for 10 seconds. The measurement was performed on each test piece (five), and an average value thereof was calculated.

(10) Average Coefficient of Dynamic Friction (MIU) of Nonwoven Fabric for Skin Care Products

Ten test pieces each having a width of 80 mm and a length of 120 mm were prepared from the sample of the nonwoven fabric for skin care products (for MD direction measurement and CD direction measurement: five sheets each). Next, for five of these ten test pieces, the coefficient of friction was measured in the MD direction of the nonwoven fabric for skin care products in the manufacturing process of the nonwoven fabric for skin care products. For the remaining five of these ten test pieces, the coefficient of friction was measured in the CD direction of the nonwoven fabric for skin care products in the manufacturing process of the nonwoven fabric for skin care products. Specifically, for evaluation in the MD direction, the test piece was attached to a roughness/friction tester (KES-SESRU manufactured by Kato Tech Co., Ltd.), the average coefficient of dynamic friction (MIU) was measured while the FR-GU terminal moved by 20 mm, with SENS as H (High), roughness static load as 10.0 gf (98 mN), SPEED as 1.0 mm/sec, and measurement mode as one-way, and the average value of the total of 5 measured values obtained was taken as the coefficient of friction in the MD direction. Furthermore, for evaluation in the CD direction, the test piece was attached to a roughness/friction tester (KES-SESRU manufactured by Kato Tech Co., Ltd.), the average coefficient of dynamic friction (MIU) was measured while the FR-GU terminal moved by 20 mm, with SENS as H (High), roughness static load as 10.0 gf (98 mN), SPEED as 1.0 mm/sec, and measurement mode as one-way, and the average value of the total of 5 measured values obtained was taken as the coefficient of friction in the CD direction. The average value of the coefficients of friction in each direction obtained was taken as the coefficient of friction of the nonwoven fabric for skin care products.

(11) Surface Characteristics of Nonwoven Fabric for Skin Care Products

Under the same conditions as in the above (10), the values of the variation in the average coefficient of dynamic friction (MMD) and the standard deviation of the average surface roughness (SMD) of the nonwoven fabric for skin care products were measured.

Next, the surface characteristics of the nonwoven fabric for skin care products were obtained by the following formula (2), from the value of the average coefficient of dynamic friction (MIU) of the nonwoven fabric for skin care products measured in the above (10), and the values of the variation in the average coefficient of dynamic friction (MMD) of the nonwoven fabric for skin care products measured in the above and the standard deviation of the average surface roughness (SMD).

Surface characteristics=MIU/(MMD×SMD) (2)

(12) Strength at 10% Elongation when Wet of Nonwoven Fabric for Skin Care Products

The measurement was performed according to JIS L1913: 2010.6.3.2. Ten test pieces each having a width of 25 mm and a length of 150 mm were prepared from the sample of the nonwoven fabric for skin care products (for MD direction measurement and CD direction measurement: five sheets each). Next, for five of these ten test pieces, the strength at 10% elongation when wet was measured in the MD direction of the nonwoven fabric for skin care products in the manufacturing process of the nonwoven fabric for skin care products. For the remaining five of these ten test pieces, the strength at 10% elongation when wet was measured in the CD direction of the nonwoven fabric for skin care products in the manufacturing process of the nonwoven fabric for skin care products. Specifically, for evaluation in the MD direction, the test piece was impregnated in 100 mL of distilled water (water temperature: 20° C.) for 10 minutes, and after the test piece was taken out from the distilled water, the test piece was immediately attached to a constant-speed extension-type tensile tester (“AG-50 kNG” manufactured by Shimadzu Corporation) to measure the tensile strength. A load was applied until the test piece was cut with a grip interval of 100 mm in the length direction of the test piece and a tensile speed of 200 ram/min, the strength when the test piece was elongated by 10% was read from a stress-strain curve, and the average value of the total of 5 measured values obtained was taken as the strength at 10% elongation when wet in the MD direction. Furthermore, for evaluation in the CD direction, the test piece was impregnated in 100 mL of distilled water (water temperature: 20° C.) for 10 minutes, and after the test piece was taken out from the distilled water, the test piece was immediately attached to a constant-speed extension-type tensile tester (“AG-50 kNG” manufactured by Shimadzu Corporation) to measure the tensile strength. A load was applied until the test piece was cut with a grip interval of 100 mm in the length direction of the test piece and a tensile speed of 200 ram/min, the strength when the test piece was elongated by 10% was read from the stress-strain curve, and the average value of the total of 5 measured values obtained was taken as the strength at 10% elongation when wet in the CD direction. The average value of the strength at 10% elongation in each direction obtained was taken as the strength at 10% elongation of the nonwoven fabric for skin care products.

(13) Monitor Evaluation (Reference Information)

1) First Time

[Face Mask]

The nonwoven fabric for skin care products obtained in Examples 1 to 16 and Comparative Examples 1 to 9 was punched into a mask shape to prepare a nonwoven fabric for face masks, and the nonwoven fabric for face masks was immersed in a lotion (“lotion/moist type for sensitive skin” manufactured by MUJI) to obtain a face mask containing 700% by mass of the lotion to a total mass of the nonwoven fabric for face masks. Next, the adhesion and soft touch to the skin of this face mask were evaluated by female panelists of 10 people on a scale of 10 points with an absolute evaluation of each person, and evaluated according to the following criteria from the average point of 10 people (rounded off to a nearest whole number).
A: 9 to 10 points,
B: 6 to 8 points,
C: 3 to 5 points, and
D: 0 to 2 points.

[Cleansing Sheet]

The nonwoven fabric for skin care products obtained in Examples 1 to 16 and Comparative Examples 1 to 9 was cut into a width of 70 mm and a length of 55 mm, and the cut nonwoven fabric was immersed in a cleansing agent (“White Cleansing Water” manufactured by Kose Cosmeport Co., Ltd.) to obtain a cleansing sheet containing 700% by mass of the cleansing agent to a total mass of the cut nonwoven fabric. Next, the cleansing sheet was evaluated by female panelists of 10 people on a scale of 10 points with an absolute evaluation of each person regarding the wiping properties against cosmetics (“Benefique Theoty Smart Liquid Eyeliner” manufactured by Shiseido Co., Ltd.) attached to the eyeliner, the soft touch to the skin, and the handling properties during wiping, and evaluated according to the following criteria from an average point of 10 people (rounded off to a nearest whole number).
A: 9 to 10 points,
B: 6 to 8 points,
C: 3 to 5 points, and
D: 0 to 2 points.

2) Second Time

[Face Mask]

The nonwoven fabric for skin care products obtained in Examples 1 to 31 and Comparative Examples 1 to 9 was punched into a mask shape to prepare a nonwoven fabric for face masks, and the nonwoven fabric for face masks was immersed in a lotion (“lotion/moist type for sensitive skin” manufactured by MUJI) to obtain a face mask containing 700% by mass of the lotion to the total mass of the nonwoven fabric for face masks. Next, the adhesion and soft touch to the skin of this face mask were evaluated by female panelists of 10 people on a scale of 10 points with an absolute evaluation of each person, and evaluated according to the following criteria from an average point of 10 people (rounded off to a nearest whole number).
A: 9 to 10 points,
B: 7 to 8 points,
C: 5 to 6 points,
D: 3 to 4 points, and
E: 0 to 2 points.

[Cleansing Sheet]

The nonwoven fabric for skin care products obtained in Examples 1 to 31 and Comparative Examples 1 to 9 was cut into a width of 70 mm and a length of 55 mm, and the cut nonwoven fabric was immersed in the cleansing agent (“White Cleansing Water” manufactured by Kose Cosmeport Co., Ltd.) to obtain a cleansing sheet containing 700% by mass of the cleansing agent to a total mass of the cut nonwoven fabric. Next, the cleansing sheet was evaluated by female panelists of 10 people on a scale of 10 points with an absolute evaluation of each person regarding the wiping properties against cosmetics (“Benefique Theoty Smart Liquid Eyeliner” manufactured by Shiseido Co., Ltd.) attached to the eyeliner, the soft touch to the skin, and the handling properties during wiping, and evaluated according to the following criteria from an average point of 10 people (rounded off to a nearest whole number).
A: 9 to 10 points,
B: 7 to 8 points,
C: 5 to 6 points,
D: 3 to 4 points, and
E: 0 to 2 points.

### Example 1

Acrylic fibers (single fiber diameter: 9 μm, flatness: 1.2, recess depth ratio: 5.6%, tensile strength: 3.0 cN/dtex, variation in single fiber diameter: 8%) of 50% by mass and rayon fibers (single fiber diameter: 12 μm) of 50% by mass were subjected to an opener step, a carding step, and a wrapping step to obtain a web. Next, the web was entangled with the high-pressure water flow to obtain a nonwoven fabric for skin care products having a basis weight of 50 g/m². A high-pressure water flow treatment was performed by injecting columnar water flows from the front and back surfaces of this web for three times in total (3 MPa, 10 MPa, and 4 MPa). The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 2

A nonwoven fabric for skin care products was obtained in the same manner as in Example 1 except that the single fiber diameter of the acrylic fibers contained in the nonwoven fabric for skin care products of Example 1 was changed to 12 μm. The average single fiber diameter of all fibers contained in the nonwoven fabric for skin care products was 12 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 3

A nonwoven fabric for skin care products was obtained in the same manner as in Example 1 except that the single fiber diameter of the acrylic fibers contained in the nonwoven fabric for skin care products of Example 1 was changed to 20 μm. The average single fiber diameter of all fibers contained in the nonwoven fabric for skin care products was 15 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 4

A nonwoven fabric for skin care products was obtained in the same manner as in Example 1 except that 50% by mass of acrylic fibers (single fiber diameter: 20 μm, flatness: 1.2, recess depth ratio: 5.6%, tensile strength: 3.0 cN/dtex, variation in single fiber diameter: 8%), 30% by mass of rayon fibers (single fiber diameter: 14 μm), and 20% by mass of polyethylene terephthalate (PET) fibers (single fiber diameter: 25 μm) were used. The average single fiber diameter of all fibers contained in the nonwoven fabric for skin care products was 18 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 5

A nonwoven fabric for skin care products was obtained in the same manner as in Example 1 except that the flatness of the acrylic fibers contained in the nonwoven fabric for skin care products of Example 1 was changed to 1.4. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 6

A nonwoven fabric for skin care products was obtained in the same manner as in Example 1 except that the flatness of the acrylic fibers contained in the nonwoven fabric for skin care products of Example 1 was changed to 1.5. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 7

A nonwoven fabric for skin care products was obtained in the same manner as in Example 1 except that the recess depth ratio of the acrylic fibers contained in the nonwoven fabric for skin care products of Example 1 was changed to 7.2%. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 8

A nonwoven fabric for skin care products was obtained in the same manner as in Example 1 except that the recess depth ratio of the acrylic fibers contained in the nonwoven fabric for skin care products of Example 1 was changed to 9.0%. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 9

A nonwoven fabric for skin care products was obtained in the same manner as in Example 1 except that the content of the acrylic fibers contained in the nonwoven fabric for skin care products of Example 1 was changed to 30% by mass, and the content of the rayon fibers was changed to 70% by mass. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 10

A nonwoven fabric for skin care products was obtained in the same manner as in Example 1 except that the content of the acrylic fibers contained in the nonwoven fabric for skin care products of Example 1 was changed to 70% by mass, and the content of the rayon fibers was changed to 30% by mass. The average single fiber diameter of all fibers contained in the nonwoven fabric for skin care products was 9 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 11

A nonwoven fabric for skin care products was obtained in the same manner as in Example 1 except that the tensile strength of the acrylic fibers contained in the nonwoven fabric for skin care products of Example 1 was changed to 3.5 cN/dtex. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 12

A nonwoven fabric for skin care products was obtained in the same manner as in Example 1 except that the acrylic fibers contained in the nonwoven fabric for skin care products of Example 1 were changed to nylon 6 (N6) fibers (single fiber diameter: 9 μm, flatness: 1.1, recess depth ratio: 4.0%, tensile strength: 4.9 cN/dtex, variation in single fiber diameter: 9%). The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 13

A nonwoven fabric for skin care products was obtained in the same manner as in Example 1 except that the acrylic fibers contained in the nonwoven fabric for skin care products of Example 1 were changed to the polyethylene terephthalate fibers (single fiber diameter: 9 μm, flatness: 1.3, recess depth ratio: 5.1%, tensile strength: 5.8 cN/dtex, variation in single fiber diameter: 6%). The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 14

A nonwoven fabric for skin care products was obtained in the same manner as in Example 1 except that the variation in the single fiber diameter of the acrylic fibers contained in the nonwoven fabric for skin care products of Example 1 was changed to 12%. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 15

A nonwoven fabric for skin care products was obtained in the same manner as in Example 1 except that the variation in the single fiber diameter of the acrylic fibers contained in the nonwoven fabric for skin care products of Example 1 was changed to 18%. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 16

A nonwoven fabric for skin care products was obtained in the same manner as in Example 1 except that the variation in the single fiber diameter of the acrylic fibers contained in the nonwoven fabric for skin care products of Example 1 was changed to 23%. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 17

A nonwoven fabric for skin care products was obtained in the same manner as in Example 1 except that the high-pressure water flow treatment described in Example 1 was changed to five times in total (3 MPa, 6 MPa, 9 MPa, 12 MPa, 13 MPa). The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 1, and physical properties and the like of the nonwoven fabric for skin care products were summarized in Table 2.

### Example 18

Acrylic fibers (single fiber diameter: 9 μm, flatness: 1.2, recess depth ratio: 5.6%, tensile strength: 3.0 cN/dtex, variation in single fiber diameter: 8%) of 100% by mass were subjected to the opener step, the carding step, and the wrapping step to obtain the web of the first layer. Furthermore, 30% by mass of the acrylic fibers and 70% by mass of the rayon fibers (single fiber diameter: 12 μm, variation in single fiber diameter: 11%) were subjected to the opener step, the carding step, and the wrapping step to obtain the web of the second layer. Next, the web of the first layer and the web of the second layer were laminated and entangled with the high-pressure water flow to obtain a nonwoven fabric for skin care products having a two-layer laminated composition (first layer/second layer) with a basis weight of 50 g/m². A high-pressure water flow treatment was performed by injecting columnar water flows from the front and back surfaces of this web for three times in total (3 MPa, 10 MPa, and 4 MPa). Here, the basis weight of the first layer was 20 g/m², and the basis weight of the second layer was 30 g/m². The content of the acrylic fibers contained in the nonwoven fabric for skin care products was 58% by mass, and the content of the rayon fibers was 42% by mass. Furthermore, the average single fiber diameter of all fibers contained in the second layer was 10 μm, the variation in the single fiber diameter of all fibers contained in the second layer was 16%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 19

A nonwoven fabric for skin care products having the two-layer laminated composition (first layer/second layer) with a basis weight of 50 g/m² was obtained in the same manner as in Example 18 except that the high-pressure water flow treatment described in Example 18 was changed to five times in total (3 MPa, 6 MPa, 9 MPa, 12 MPa, and 13 MPa). Here, the basis weight of the first layer was 20 g/m², and the basis weight of the second layer was 30 g/m². The content of the acrylic fibers contained in the nonwoven fabric for skin care products was 58% by mass, and the content of the rayon fibers was 42% by mass. Furthermore, the average single fiber diameter of all fibers contained in the second layer was 10 μm, the variation in the single fiber diameter of all fibers contained in the second layer was 16%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 20

The acrylic fibers (single fiber diameter: 9 μm, flatness: 1.2, recess depth ratio: 5.6%, tensile strength: 3.0 cN/dtex, variation in single fiber diameter: 8%) of 70% by mass and the rayon fibers (single fiber diameter: 12 μm, variation in single fiber diameter: 11%) of 30% by mass were subjected to the opener step, the carding step, and the wrapping step to obtain the web of the first layer.

Furthermore, 40% by mass of the acrylic fibers and 60% by mass of the rayon fibers were subjected to the opener step, the carding step, and the wrapping step to obtain the web of the second layer. Next, the web of the first layer and the web of the second layer were laminated and entangled with the high-pressure water flow to obtain a nonwoven fabric for skin care products having a two-layer laminated composition (first layer/second layer) with a basis weight of 50 g/m². The high-pressure water flow treatment was performed by injecting the columnar water flows from the front and back surfaces of this web for five times in total (3 MPa, 6 MPa, 9 MPa, 12 MPa, 13 MPa). Here, the basis weight of the first layer was 20 g/m², and the basis weight of the second layer was 30 g/m². The content of the acrylic fibers contained in the nonwoven fabric for skin care products was 52% by mass, and the content of the rayon fibers was 48% by mass. Furthermore, the average single fiber diameter of all fibers contained in the second layer was 10 μm, the variation in the single fiber diameter of all fibers contained in the second layer was 16%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 21

The acrylic fibers (single fiber diameter: 9 μm, flatness: 1.2, recess depth ratio: 5.6%, tensile strength: 3.0 cN/dtex, variation in single fiber diameter: 8%) of 50% by mass and the rayon fibers (single fiber diameter: 12 μm, variation in single fiber diameter: 11%) of 50% by mass were subjected to the opener step, the carding step, and the wrapping step to obtain the web of the first layer. Furthermore, 20% by mass of the acrylic fibers and 80% by mass of the rayon fibers were subjected to the opener step, the carding step, and the wrapping step to obtain the web of the second layer. Next, the web of the first layer and the web of the second layer were laminated and entangled with the high-pressure water flow to obtain a nonwoven fabric for skin care products having a two-layer laminated composition (first layer/second layer) with a basis weight of 50 g/m². The high-pressure water flow treatment was performed by injecting the columnar water flows from the front and back surfaces of this web for five times in total (3 MPa, 6 MPa, 9 MPa, 12 MPa, 13 MPa). Here, the basis weight of the first layer was 20 g/m², and the basis weight of the second layer was 30 g/m². The content of the acrylic fibers contained in the nonwoven fabric for skin care products was 32% by mass, and the content of the rayon fibers was 68% by mass. Furthermore, the average single fiber diameter of all fibers contained in the second layer was 11 μm, the variation in the single fiber diameter of all fibers contained in the second layer was 14%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 22

The acrylic fibers (single fiber diameter: 9 μm, flatness: 1.2, recess depth ratio: 5.6%, tensile strength: 3.0 cN/dtex, variation in single fiber diameter: 8%) of 40% by mass and the rayon fibers (single fiber diameter: 12 μm, variation in single fiber diameter: 11%) of 60% by mass were subjected to the opener step, the carding step, and the wrapping step to obtain the web of the first layer. Furthermore, 60% by mass of the acrylic fibers and 40% by mass of the rayon fibers were subjected to the opener step, the carding step, and the wrapping step to obtain the web of the second layer. Next, the web of the first layer and the web of the second layer were laminated and entangled with the high-pressure water flow to obtain a nonwoven fabric for skin care products having a two-layer laminated composition (first layer/second layer) with a basis weight of 50 g/m². The high-pressure water flow treatment was performed by injecting the columnar water flows from the front and back surfaces of this web for five times in total (3 MPa, 6 MPa, 9 MPa, 12 MPa, 13 MPa). Here, the basis weight of the first layer was 20 g/m², and the basis weight of the second layer was 30 g/m². The content of the acrylic fibers contained in the nonwoven fabric for skin care products was 52% by mass, and the content of the rayon fibers was 48% by mass. Furthermore, the average single fiber diameter of all fibers contained in the second layer was 10 μm, the variation in the single fiber diameter of all fibers contained in the second layer was 14%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 23

A nonwoven fabric for skin care products having the two-layer laminated composition (first layer/second layer) with a basis weight of 50 g/m² was obtained in the same manner as in Example 19 except that the variation in the single fiber diameter of the acrylic fibers contained in the nonwoven fabric for skin care products of Example 19 was changed to 9%. Here, the basis weight of the first layer was 20 g/m², and the basis weight of the second layer was 30 g/m². The content of the acrylic fibers contained in the nonwoven fabric for skin care products was 58% by mass, and the content of the rayon fibers was 42% by mass. Furthermore, the average single fiber diameter of all fibers contained in the second layer was 10 μm, the variation in the single fiber diameter of all fibers contained in the second layer was 16%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 24

A nonwoven fabric for skin care products having the two-layer laminated composition (first layer/second layer) with a basis weight of 50 g/m² was obtained in the same manner as in Example 19 except that the variation in the single fiber diameter of the acrylic fibers contained in the nonwoven fabric for skin care products of Example 19 was changed to 10%. Here, the basis weight of the first layer was 20 g/m², and the basis weight of the second layer was 30 g/m². The content of the acrylic fibers contained in the nonwoven fabric for skin care products was 58% by mass, and the content of the rayon fibers was 42% by mass. Furthermore, the average single fiber diameter of all fibers contained in the second layer was 10 μm, the variation in the single fiber diameter of all fibers contained in the second layer was 16%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 25

A nonwoven fabric for skin care products having the two-layer laminated composition (first layer/second layer) with a basis weight of 50 g/m² was obtained in the same manner as in Example 19 except that the variation in the single fiber diameter of the acrylic fibers contained in the nonwoven fabric for skin care products of Example 19 was changed to 11%. Here, the basis weight of the first layer was 20 g/m², and the basis weight of the second layer was 30 g/m². The content of the acrylic fibers contained in the nonwoven fabric for skin care products was 58% by mass, and the content of the rayon fibers was 42% by mass. Furthermore, the average single fiber diameter of all fibers contained in the second layer was 10 μm, the variation in the single fiber diameter of all fibers contained in the second layer was 16%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 26

The web of the first layer was prepared in the same manner as in Example 19. Furthermore, 5% by mass of acrylic fibers (single fiber diameter: 9 μm, flatness: 1.2, recess depth ratio: 5.6%, tensile strength: 3.0 cN/dtex, variation in single fiber diameter: 8%) and 55% by mass of the rayon fibers (single fiber diameter: 12 μm, variation in single fiber diameter: 11%), and 40% by mass of the polyethylene terephthalate fibers (single fiber diameter: 22 μm, variation in single fiber diameter: 13%) were subjected to the opener step, the carding step, and the wrapping step to obtain the web of the second layer. Next, the web of the first layer and the web of the second layer were laminated and entangled with the high-pressure water flow to obtain a nonwoven fabric for skin care products having a two-layer laminated composition (first layer/second layer) with a basis weight of 50 g/m². The high-pressure water flow treatment was performed by injecting the columnar water flows from the front and back surfaces of this web for five times in total (3 MPa, 6 MPa, 9 MPa, 12 MPa, and 13 MPa). Here, the basis weight of the first layer was 20 g/m², and the basis weight of the second layer was 30 g/m². The content of the acrylic fibers contained in the nonwoven fabric for skin care products was 43% by mass, the content of the rayon fibers was 33% by mass, and the content of the polyethylene terephthalate fibers was 24% by mass. Furthermore, the average single fiber diameter of all fibers contained in the second layer was 13 μm, the variation in the single fiber diameter of all fibers contained in the second layer was 27%, and the average single fiber diameter of all fibers contained in the nonwoven fabric for skin care products was 12 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 27

The web of the first layer was prepared in the same manner as in Example 19. Furthermore, 60% by mass of the rayon fibers (single fiber diameter: 14 μm, variation in single fiber diameter: 11%) and 40% by mass of the polyethylene terephthalate fibers (single fiber diameter: 22 μm, variation in single fiber diameter: 13%) were subjected to the opener step, the carding step, and the wrapping step to obtain the web of the second layer. Next, the web of the first layer and the web of the second layer were laminated and entangled with the high-pressure water flow to obtain a nonwoven fabric for skin care products having a two-layer laminated composition (first layer/second layer) with a basis weight of 50 g/m². The high-pressure water flow treatment was performed by injecting the columnar water flows from the front and back surfaces of this web for five times in total (3 MPa, 6 MPa, 9 MPa, 12 MPa, and 13 MPa). Here, the basis weight of the first layer was 20 g/m², and the basis weight of the second layer was 30 g/m². The content of the acrylic fibers contained in the nonwoven fabric for skin care products was 40% by mass, the content of the rayon fibers was 36% by mass, and the content of the polyethylene terephthalate fibers was 24% by mass. Furthermore, the average single fiber diameter of all fibers contained in the second layer was 15 μm, the variation in the single fiber diameter of all fibers contained in the second layer was 19%, and the average single fiber diameter of all fibers contained in the nonwoven fabric for skin care products was 12 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 28

The web of the first layer was prepared in the same manner as in Example 19. Furthermore, 50% by mass of the rayon fibers (single fiber diameter: 15 μm, variation in single fiber diameter: 11%) and 50% by mass of the polyethylene terephthalate fibers (single fiber diameter: 22 μm, variation in single fiber diameter: 13%) were subjected to the opener step, the carding step, and the wrapping step to obtain the web of the second layer. Next, the web of the first layer and the web of the second layer were laminated and entangled with the high-pressure water flow to obtain a nonwoven fabric for skin care products having a two-layer laminated composition (first layer/second layer) with a basis weight of 50 g/m². The high-pressure water flow treatment was performed by injecting the columnar water flows from the front and back surfaces of this web for five times in total (3 MPa, 6 MPa, 9 MPa, 12 MPa, and 13 MPa). Here, the basis weight of the first layer was 20 g/m², and the basis weight of the second layer was 30 g/m². The content of the acrylic fibers contained in the nonwoven fabric for skin care products was 40% by mass, the content of the rayon fibers was 30% by mass, and the content of the polyethylene terephthalate fibers was 30% by mass. Furthermore, the average single fiber diameter of all fibers contained in the second layer was 17 μm, the variation in the single fiber diameter of all fibers contained in the second layer was 17%, and the average single fiber diameter of all fibers contained in the nonwoven fabric for skin care products was 12 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 29

A nonwoven fabric for skin care products having the two-layer laminated composition (first layer/second layer) with a basis weight of 50 g/m² was obtained in the same manner as in Example 27 except that the single fiber diameter of the rayon fiber contained in the second layer of Example 27 was changed to 10 μm. Here, the basis weight of the first layer was 20 g/m², and the basis weight of the second layer was 30 g/m². The content of the acrylic fibers contained in the nonwoven fabric for skin care products was 40% by mass, the content of the rayon fibers was 36% by mass, and the content of the polyethylene terephthalate fibers was 24% by mass. Furthermore, the average single fiber diameter of all fibers contained in the second layer was 11 μm, the variation in the single fiber diameter of all fibers contained in the second layer was 31%, and the average single fiber diameter of all fibers contained in the nonwoven fabric for skin care products was 11 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 30

A nonwoven fabric for skin care products having the two-layer laminated composition (first layer/second layer) with a basis weight of 50 g/m² was obtained in the same manner as in Example 29 except that the single fiber diameter of the polyethylene terephthalate fiber contained in the second layer of Example 29 was changed to 25 μm. Here, the basis weight of the first layer was 20 g/m², and the basis weight of the second layer was 30 g/m². The content of the acrylic fibers contained in the nonwoven fabric for skin care products was 40% by mass, the content of the rayon fibers was 36% by mass, and the content of the polyethylene terephthalate fibers was 24% by mass. Furthermore, the average single fiber diameter of all fibers contained in the second layer was 11 μm, the variation in the single fiber diameter of all fibers contained in the second layer was 36%, and the average single fiber diameter of all fibers contained in the nonwoven fabric for skin care products was 11 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present Example was summarized in Table 5, and physical properties and the like of the nonwoven fabric for skin care products were summarized in Table 6.

### Example 31

The acrylic fibers (single fiber diameter: 9 μm, flatness: 1.2, recess depth ratio: 5.6%, tensile strength: 3.0 cN/dtex, variation in single fiber diameter: 8%) of 70% by mass and the rayon fibers (single fiber diameter: 12 μm, variation in single fiber diameter: 11%) of 30% by mass were subjected to the opener step, the carding step, and the wrapping step to obtain the web of the first layer. Furthermore, 20% by mass of the acrylic fibers and 80% by mass of the rayon fibers were subjected to the opener step, the carding step, and the wrapping step to obtain the web of the second layer. Next, the web of the first layer was laminated on the front and back surfaces of the web of the second layer and entangled with the high-pressure water flow to obtain a nonwoven fabric for skin care products having a three-layer laminated composition (first layer/second layer/first layer) with a basis weight of 50 g/m². The high-pressure water flow treatment was performed by injecting the columnar water flows from the front and back surfaces of this web for five times in total (3 MPa, 6 MPa, 9 MPa, 12 MPa, and 13 MPa). Here, the basis weight of the first layer was 30 g/m², and the basis weight of the second layer was 20 g/m². The content of the acrylic fibers contained in the nonwoven fabric for skin care products was 50% by mass, and the content of the rayon fibers was 50% by mass. Furthermore, the average single fiber diameter of all fibers contained in the second layer was 11 μm, the variation in the single fiber diameter of all fibers contained in the second layer was 14%, and the average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, a nonwoven fabric composition of the nonwoven fabric for skin care products of the present example was summarized in Table 5, and physical properties and the like of the nonwoven fabric for skin care products were summarized in Table 6.

### Comparative Example 1

An attempt was made to prepare a nonwoven fabric for skin care products in the same manner as in Example 1 except that the single fiber diameter of the acrylic fibers contained in the nonwoven fabric for skin care products of Example 1 was changed to 0.8 μm. However, in the carding process, waste cotton due to thread breakage and winding around the card clothing frequently occurred, so that a web could not be obtained, and a nonwoven fabric for skin care products could not be produced.

### Comparative Example 2

A nonwoven fabric for skin care products was obtained in the same manner as in Example 1 except that the single fiber diameter of the acrylic fibers contained in the nonwoven fabric for skin care products of Example 1 was changed to 25 μm. The average single fiber diameter of all fibers contained in the nonwoven fabric for skin care products was 15 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

### Comparative Example 3

A nonwoven fabric for skin care products was obtained in the same manner as in Example 1 except that the single fiber diameter of the acrylic fibers contained in the nonwoven fabric for skin care products of Example 1 was changed to 3 μm. The average single fiber diameter of all fibers contained in the nonwoven fabric for skin care products was 4 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

### Comparative Example 4

A nonwoven fabric for skin care products was obtained in the same manner as in Example 1 except that 50% by mass of acrylic fibers (single fiber diameter: 20 μm, flatness: 1.2, recess depth ratio: 5.6%, tensile strength: 3.0 cN/dtex, variation in single fiber diameter: 8%), 30% by mass of the rayon fibers (single fiber diameter: 23 μm), and 20% by mass of the polyethylene terephthalate fibers (single fiber diameter: 25 μm) were used. The average single fiber diameter of all fibers contained in the nonwoven fabric for skin care products was 21 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

### Comparative Example 5

A nonwoven fabric for skin care products was obtained in the same manner as in Example 1 except that the flatness of the acrylic fibers contained in the nonwoven fabric for skin care products of Example 1 was changed to 1.8. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

### Comparative Example 6

A nonwoven fabric for skin care products was obtained in the same manner as in Example 1 except that the recess depth ratio of the acrylic fibers contained in the nonwoven fabric for skin care products of Example 1 was changed to 0.5%. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

### Comparative Example 7

A nonwoven fabric for skin care products was obtained in the same manner as in Example 1 except that the recess depth ratio of the acrylic fibers contained in the nonwoven fabric for skin care products of Example 1 was changed to 15.0%. The average single fiber diameter of all fibers contained in the nonwoven fabric for skin care products was 10 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

### Comparative Example 8

A nonwoven fabric for skin care products was obtained in the same manner as in Example 1 except that the content of the acrylic fibers contained in the nonwoven fabric for skin care products of Example 1 was changed to 20% by mass, and the content of the rayon fibers was changed to 80% by mass. The average single fiber diameter of all fibers contained in the nonwoven fabric for skin care products was 11 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

### Comparative Example 9

A nonwoven fabric for skin care products was obtained in the same manner as in Example 1 except that the content of the acrylic fibers contained in the nonwoven fabric for skin care products of Example 1 was changed to 80% by mass, and the content of the rayon fibers was changed to 20% by mass. The average single fiber diameter of all fibers contained in the nonwoven fabric for skin care products was 9 μm.

Next, the nonwoven fabric for skin care products was punched into a mask shape to obtain a face mask. Furthermore, separately from the face mask, the nonwoven fabric for skin care products was cut into a width of 70 mm and a length of 55 mm to obtain a cleansing sheet.

Here, the nonwoven fabric composition of the nonwoven fabric for skin care products of the present Comparative Example was summarized in Table 3, and the physical properties and the like of the nonwoven fabric for skin care products were summarized in Table 4.

Here, the nonwoven fabric for skin care products of Comparative Example 1 could not be produced. This is presumed to be because the single fiber diameter of the acrylic fibers used in the nonwoven fabric for skin care products of Comparative Example 1 is smaller than the single fiber diameter of the acrylic fibers used in the nonwoven fabric for skin care products (the nonwoven fabric 1) of Example 1.

Furthermore, the adhesion of the face mask and the wiping properties of the cleansing sheet of Comparative Example 2 were inferior to the adhesion of the face mask and the wiping properties of the cleansing sheet of Example 1. This is presumed to be because the single fiber diameter of the acrylic fibers contained in the nonwoven fabric for skin care products used in the face mask and the cleansing sheet of Comparative Example 2 is larger than the single fiber diameter of the acrylic fibers contained in the nonwoven fabric 1.

Furthermore, the adhesion of the face mask and the wiping properties and handling properties of the cleansing sheet of Comparative Example 3 were inferior to the adhesion of the face mask and the wiping properties and handling properties of the cleansing sheet of Example 1. This is presumed to be because the average single fiber diameter of all fibers contained in the nonwoven fabric for skin care products used in the face mask and the cleansing sheet of Comparative Example 3 is smaller than the average single fiber diameter of all fibers contained in the nonwoven fabric 1.

Furthermore, the adhesion of the face mask and the wiping properties of the cleansing sheet of Comparative Example 4 were inferior to the adhesion of the face mask and the wiping properties of the cleansing sheet of Example 1. This is presumed to be because the average single fiber diameter of all fibers contained in the nonwoven fabric for skin care products used in the face mask and the cleansing sheet of Comparative Example 4 is larger than the average single fiber diameter of all fibers contained in the nonwoven fabric 1.

Furthermore, the adhesion of the face mask and the wiping properties of the cleansing sheet of Comparative Example 5 were slightly inferior to the adhesion of the face mask and the wiping properties of the cleansing sheet of Example 1. Further, the soft touch to the skin of the face mask and the cleansing sheet of Comparative Example 5 was inferior to the soft touch to the skin of the face mask and the cleansing sheet of Example 1. This is presumed to be because the flatness of the acrylic fibers contained in the nonwoven fabric for skin care products used in the face mask and the cleansing sheet of Comparative Example 5 is larger than the flatness of the acrylic fibers contained in the nonwoven fabric 1.

Furthermore, the adhesion of the face mask and the wiping properties of the cleansing sheet of Comparative Example 6 were inferior to the adhesion of the face mask and the wiping properties of the cleansing sheet of Example 1. This is presumed to be because the recess depth ratio in the acrylic fiber contained in the nonwoven fabric for skin care products used in the face mask and the cleansing sheet of Comparative Example 6 is smaller than the recess depth ratio in the acrylic fiber contained in the nonwoven fabric 1.

Furthermore, the soft touch to the skin of the face mask and the cleansing sheet of Comparative Example 7 was inferior to the soft touch to the skin of the face mask and the cleansing sheet of Example 1. This is presumed to be because the recess depth ratio in the acrylic fiber contained in the nonwoven fabric for skin care products used in the face mask and the cleansing sheet of Comparative Example 7 is larger than the recess depth ratio in the acrylic fiber contained in the nonwoven fabric 1.

Furthermore, the adhesion of the face mask and the wiping properties of the cleansing sheet of Comparative Example 8 were inferior to the adhesion of the face mask and the wiping properties of the cleansing sheet of Example 1. Further, the soft touch to the skin of the face mask and the cleansing sheet of Comparative Example 8 was inferior to the soft touch to the skin of the face mask and the cleansing sheet of Example 1. This is presumed to be because the content of acrylic fibers contained in the nonwoven fabric for skin care products used in the face mask and the cleansing sheet of Comparative Example 8 is smaller than the content of acrylic fibers contained in the nonwoven fabric 1.

Furthermore, the adhesion of the face mask and the wiping properties of the cleansing sheet of Comparative Example 9 were inferior to the adhesion of the face mask and the wiping properties of the cleansing sheet of Example 1. This is presumed to be because the content of acrylic fibers contained in the nonwoven fabric for skin care products used in the face mask and the cleansing sheet of Comparative Example 9 is larger than the content of acrylic fibers contained in the nonwoven fabric 1.

TABLE 1-1

| | | | | Exam- | Exam- | Exam- | Exam- | Exam- | Exam- | Exam- | Exam- | Exam- |
|---|---|---|---|---|---|---|---|---|---|---|---|---|
| | | | Unit | ple 1 | ple 2 | ple 3 | ple 4 | ple 5 | ple 6 | ple 7 | ple 8 | ple 9 |
| Nonwoven | Synthetic | Materials | — | Acrylic | Acrylic | Acrylic | Acrylic | Acrylic | Acrylic | Acrylic | Acrylic | Acrylic |
| fabric | fibers | Single fiber | μm | 9 | 12 | 20 | 20 | 9 | 9 | 9 | 9 | 9 |
| composition | | diameter | | | | | | | | | | |
| | | Variation in | % | 8 | 8 | 8 | 8 | 8 | 8 | 8 | 8 | 8 |
| | | fiber diameter | | | | | | | | | | |
| | | Flatness | — | 1.2 | 1.2 | 1.2 | 1.2 | 1.4 | 1.5 | 1.2 | 1.2 | 1.2 |
| | | Recess depth | % | 5.6 | 5.6 | 5.6 | 5.6 | 5.6 | 5.6 | 7.2 | 9.0 | 5.6 |
| | | ratio | | | | | | | | | | |
| | | Tensile | — | 3.0 | 3.0 | 3.0 | 3.0 | 3.0 | 3.0 | 3.0 | 3.0 | 3.0 |
| | | strength | | | | | | | | | | |
| | | Content | Mass % | 50 | 50 | 50 | 50 | 50 | 50 | 50 | 50 | 30 |
| | Cellulose | Materials | — | Rayon | Rayon | Rayon | Rayon | Rayon | Rayon | Rayon | Rayon | Rayon |
| | fibers | Single fiber | μm | 12 | 12 | 12 | 14 | 12 | 12 | 12 | 12 | 12 |
| | | diameter | | | | | | | | | | |
| | | Content | Mass % | 50 | 50 | 50 | 30 | 50 | 50 | 50 | 50 | 70 |
| | Other | Materials | — | — | — | — | PET | — | — | — | — | — |
| | Fibers | Fiber diameter | μm | — | — | — | 25 | — | — | — | — | — |
| | | Content | Mass % | — | — | — | 20 | — | — | — | — | — |

| | | | | | | | | | | |
|---|---|---|---|---|---|---|---|---|---|---|
| Average single fiber | μm | 10 | 12 | 15 | 18 | 10 | 10 | 10 | 10 | 10 |
| diameter of all fibers | | | | | | | | | | |
| Basis weight | g/cm³ | 50 | 50 | 50 | 50 | 50 | 50 | 50 | 50 | 50 |

N6: nylon 6,
PET: polyethylene terephthalate

TABLE 1-2

| | | | | Example | Example | Example | Example | Example | Example | Example | Example |
|---|---|---|---|---|---|---|---|---|---|---|---|
| | | | Unit | 10 | 11 | 12 | 13 | 14 | 15 | 16 | 17 |
| Nonwoven | Synthetic | Materials | — | Acrylic | Acrylic | N6 | PET | Acrylic | Acrylic | Acrylic | Acrylic |
| fabric | fibers | Single fiber | μm | 9 | 9 | 9 | 9 | 9 | 9 | 9 | 9 |
| composition | | diameter | | | | | | | | | |
| | | Variation in | % | 8 | 8 | 9 | 6 | 12 | 18 | 23 | 8 |
| | | fiber diameter | | | | | | | | | |
| | | Flatness | — | 1.2 | 1.2 | 1.1 | 1.3 | 1.2 | 1.2 | 1.2 | 1.2 |
| | | Recess depth | % | 5.6 | 5.6 | 4.0 | 5.1 | 5.6 | 5.6 | 5.6 | 5.6 |
| | | ratio | | | | | | | | | |
| | | Tensile | — | 3.0 | 3.5 | 4.9 | 5.8 | 3.0 | 3.0 | 3.0 | 3.0 |
| | | strength | | | | | | | | | |
| | | Content | Mass % | 70 | 50 | 50 | 50 | 50 | 50 | 50 | 50 |
| | Cellulose | Materials | — | Rayon | Rayon | Rayon | Rayon | Rayon | Rayon | Rayon | Rayon |
| | fibers | Single fiber | μm | 12 | 12 | 12 | 12 | 12 | 12 | 12 | 12 |
| | | diameter | | | | | | | | | |
| | | Content | Mass % | 30 | 50 | 50 | 50 | 50 | 50 | 50 | 50 |
| | Other | Materials | — | — | — | — | — | — | — | — | — |
| | Fibers | Fiber diameter | μm | — | — | — | — | — | — | — | — |
| | | Content | Mass % | — | — | — | — | — | — | — | — |

| | | | | | | | | | |
|---|---|---|---|---|---|---|---|---|---|
| Average single fiber | μm | 9 | 10 | 10 | 10 | 10 | 10 | 10 | 10 |
| diameter of all fibers | | | | | | | | | |
| Basis weight | g/cm² | 50 | 50 | 50 | 50 | 50 | 50 | 50 | 50 |

N6: nylon 6,
PET: polyethylene terephthalate

TABLE 2-1

| | | | | | Exam- | Exam- | Exam- | Exam- | Exam- | Exam- | Exam- | Exam- | Exam- |
|---|---|---|---|---|---|---|---|---|---|---|---|---|---|
| | | | | Unit | ple 1 | ple 2 | ple 3 | ple 4 | ple 5 | ple 6 | ple 7 | ple 8 | ple 9 |
| Physical | Average coefficient of | | | — | 0.064 | 0.061 | 0.059 | 0.058 | 0.066 | 0.067 | 0.070 | 0.073 | 0.061 |
| properties | dynamic friction (MIU) | | | | | | | | | | | | |
| | Surface characteristics | | | — | 3.3 | 3.2 | 3.1 | 2.7 | 2.5 | 2.3 | 2.6 | 2.4 | 2.9 |
| | Strength at 10% elongation | | | N/25 mm | 2.3 | 3.5 | 4.4 | 5.2 | 2.4 | 2.5 | 2.3 | 2.5 | 3.0 |
| Monitor | First | Face | Adhesion | — | A | B | C | C | A | A | A | A | B |
| evaluation | time | mask | Soft touch | — | A | A | A | B | B | C | B | C | B |
| | | | to skin | | | | | | | | | | |
| | | Cleansing | Wiping | — | A | B | C | C | A | A | A | A | B |
| | | sheet | properties | | | | | | | | | | |
| | | | Soft touch | — | A | A | A | B | B | C | B | C | B |
| | | | to skin | | | | | | | | | | |
| | | | Handling | — | A | A | A | A | A | A | A | A | A |
| | | | properties | | | | | | | | | | |
| | | | during wiping | | | | | | | | | | |
| | Second | Face | Adhesion | — | B | C | D | D | B | B | B | B | C |
| | time | mask | Soft touch | — | B | B | B | C | C | D | C | D | C |
| | | | to skin | | | | | | | | | | |
| | | Cleansing | Wiping | — | B | C | D | D | B | B | B | B | C |
| | | sheet | properties | | | | | | | | | | |
| | | | Soft touch | — | B | B | B | C | C | D | C | D | C |
| | | | to skin | | | | | | | | | | |
| | | | Handling | — | A | A | A | A | A | A | A | A | A |
| | | | properties | | | | | | | | | | |
| | | | during wiping | | | | | | | | | | |

TABLE 2-2

| | | | | | Exam- | Exam- | Exam- | Exam- | Exam- | Exam- | Exam- | Exam- |
|---|---|---|---|---|---|---|---|---|---|---|---|---|
| | | | | Unit | ple 10 | ple 11 | ple 12 | ple 13 | ple 14 | ple 15 | ple 16 | ple 17 |
| Physical | Average coefficient of | | | — | 0.066 | 0.060 | 0.059 | 0.058 | 0.063 | 0.063 | 0.062 | 0.070 |
| properties | dynamic friction (MIU) | | | | | | | | | | | |
| | Surface characteristics | | | — | 3.8 | 3.0 | 2.6 | 2.5 | 2.4 | 2.6 | 2.2 | 3.5 |
| | Strength at 10% elongation | | | N/25 mm | 2.1 | 3.1 | 5.5 | 6.0 | 2.4 | 2.5 | 2.4 | 2.0 |
| Monitor | First | Face | Adhesion | — | A | B | C | C | A | A | B | — |
| evaluation | time | mask | Soft touch | — | A | A | B | B | B | C | C | — |
| | | | to skin | | | | | | | | | |
| | | Cleansing | Wiping | — | A | B | C | C | A | A | B | — |
| | | sheet | properties | | | | | | | | | |
| | | | Soft touch | — | A | A | B | B | B | C | C | — |
| | | | to skin | | | | | | | | | |
| | | | Handling | — | A | A | A | A | A | A | A | — |
| | | | properties | | | | | | | | | |
| | | | during wiping | | | | | | | | | |
| | Second | Face | Adhesion | — | B | C | D | D | B | B | B | A |
| | time | mask | Soft touch | — | A | B | C | C | C | D | D | A |
| | | | to skin | | | | | | | | | |
| | | Cleansing | Wiping | — | B | C | D | D | B | B | B | A |
| | | sheet | properties | | | | | | | | | |
| | | | Soft touch | | A | B | C | C | C | D | D | A |
| | | | to skin | | | | | | | | | |
| | | | Handling | — | A | A | A | A | A | A | A | A |
| | | | properties | | | | | | | | | |
| | | | during wiping | | | | | | | | | |

TABLE 3-1

| | | | | Comparative | Comparative | Comparative | Comparative | Comparative |
|---|---|---|---|---|---|---|---|---|
| | | | Unit | Example 1 | Example 2 | Example 3 | Example 4 | Example 5 |
| Nonwoven | Synthetic | Materials | — | Acrylic | Acrylic | Acrylic | Acrylic | Acrylic |
| fabric | fibers | Single fiber | μm | 0.8 | 25 | 3 | 20 | 9 |
| composition | | diameter | | | | | | |
| | | Variation in | % | 8 | 8 | 8 | 8 | 8 |
| | | fiber diameter | | | | | | |
| | | Flatness | — | 1.2 | 1.2 | 1.2 | 1.2 | 1.8 |
| | | Recess depth | % | 5.6 | 5.6 | 5.6 | 5.6 | 5.6 |
| | | ratio | | | | | | |
| | | Tensile strength | — | 3.0 | 3.0 | 3.0 | 3.0 | 3.0 |
| | | Content | Mass % | 50 | 50 | 50 | 50 | 50 |
| | Cellulose | Materials | — | Rayon | Rayon | Rayon | Rayon | Rayon |
| | fibers | Single fiber | μm | 12 | 12 | 12 | 23 | 12 |
| | | diameter | | | | | | |
| | | Content | Mass % | 50 | 50 | 50 | 30 | 50 |
| | Other | Materials | — | — | — | — | PET | — |
| | Fibers | Fiber diameter | μm | — | — | — | 25 | — |
| | | Content | Mass % | — | — | — | 20 | — |

| | | | | | | |
|---|---|---|---|---|---|---|
| Average single fiber | μm | — | 15 | 4 | 21 | 10 |
| diameter of all fibers | | | | | | |
| Basis weight | g/cm² | — | 50 | 50 | 50 | 50 |

N6: nylon 6,
PET: polyethylene terephthalate

TABLE 3-2

| | | | | Comparative | Comparative | Comparative | Comparative |
|---|---|---|---|---|---|---|---|
| | | | Unit | Example 6 | Example 7 | Example 8 | Example 9 |
| Nonwoven | Synthetic | Materials | — | Acrylic | Acrylic | Acrylic | Acrylic |
| fabric | fibers | Single fiber | μm | 9 | 9 | 9 | 9 |
| composition | | diameter | | | | | |
| | | Variation in | % | 8 | 8 | 8 | 8 |
| | | fiber diameter | | | | | |
| | | Flatness | — | 1.2 | 1.2 | 1.2 | 1.2 |
| | | Recess depth | % | 0.5 | 15.0 | 5.6 | 5.6 |
| | | ratio | | | | | |
| | | Tensile strength | — | 3.0 | 3.0 | 3.0 | 3.0 |
| | | Content | Mass % | 50 | 50 | 20 | 80 |
| | Cellulose | Materials | — | Rayon | Rayon | Rayon | Rayon |
| | fibers | Single fiber | μm | 12 | 12 | 12 | 12 |
| | | diameter | | | | | |
| | | Content | Mass % | 50 | 50 | 80 | 20 |
| | Other | Materials | — | — | — | — | — |
| | Fibers | Fiber diameter | μm | — | — | — | — |
| | | Content | Mass % | — | — | — | — |

| | | | | | |
|---|---|---|---|---|---|
| Average single fiber | μm | 10 | 10 | 11 | 9 |
| diameter of all fibers | | | | | |
| Basis weight | g/cm² | 50 | 50 | 50 | 50 |

N6: nylon 6,
PET: polyethylene terephthalate

TABLE 4-1

| | | | | | Comparative | Comparative | Comparative | Comparative | Comparative |
|---|---|---|---|---|---|---|---|---|---|
| | | | | Unit | Example 1 | Example 2 | Example 3 | Example 4 | Example 5 |
| Physical | Average coefficient of | | | — | — | 0.056 | 0.070 | 0.050 | 0.075 |
| properties | dynamic friction (MIU) | | | | | | | | |
| | Surface characteristics | | | — | — | 3.2 | 3.5 | 3.4 | 2.2 |
| | Strength at 10% elongation | | | N/25 mm | — | 5.7 | 0.5 | 8.1 | 2.4 |
| Monitor | First | Face | Adhesion | — | — | D | C | D | B |
| evaluation | time | mask | Soft touch | — | — | A | A | A | D |
| | | | to skin | | | | | | |
| | | Cleansing | Wiping | — | — | D | C | D | B |
| | | sheet | properties | | | | | | |
| | | | Soft touch | — | — | A | A | A | D |
| | | | to skin | | | | | | |
| | | | Handling | — | — | A | D | A | A |
| | | | properties | | | | | | |
| | | | during wiping | | | | | | |
| | Second | Face | Adhesion | — | — | E | D | E | B |
| | time | mask | Soft touch | — | — | B | A | B | E |
| | | | to skin | | | | | | |
| | | Cleansing | Wiping | — | — | E | D | E | B |
| | | sheet | properties | | | | | | |
| | | | Soft touch | — | — | B | A | B | E |
| | | | to skin | | | | | | |
| | | | Handling | — | — | A | E | A | A |
| | | | properties | | | | | | |
| | | | during wiping | | | | | | |

TABLE 4-2

| | | | | | Comparative | Comparative | Comparative | Comparative |
|---|---|---|---|---|---|---|---|---|
| | | | | Unit | Example 6 | Example 7 | Example 8 | Example 9 |
| Physical | Average coefficient of | | | — | 0.055 | 0.076 | 0.058 | 0.064 |
| properties | dynamic friction (MIU) | | | | | | | |
| | Surface characteristics | | | — | 3.5 | 2.1 | 2.4 | 3.4 |
| | Strength at 10% elongation | | | N/25 mm | 2.3 | 2.6 | 4.3 | 1.8 |
| Monitor | First | Face | Adhesion | — | D | A | C | D |
| evaluation | time | mask | Soft touch | — | A | D | C | A |
| | | | to skin | | | | | |
| | | Cleansing | Wiping | — | D | A | C | D |
| | | sheet | properties | | | | | |
| | | | Soft touch | — | A | D | C | A |
| | | | to skin | | | | | |
| | | | Handling | — | A | A | A | A |
| | | | properties | | | | | |
| | | | during wiping | | | | | |
| | Second | Face | Adhesion | — | E | A | E | E |
| | time | mask | Soft touch | — | A | E | D | B |
| | | | to skin | | | | | |
| | | Cleansing | Wiping | — | E | A | E | E |
| | | sheet | properties | | | | | |
| | | | Soft touch | — | A | E | D | B |
| | | | to skin | | | | | |
| | | | Handling | — | A | A | A | A |
| | | | properties | | | | | |
| | | | during wiping | | | | | |

TABLE 5-1

| | | | | Example | Example | Example | Example | Example | Example | Example |
|---|---|---|---|---|---|---|---|---|---|---|
| | | | Unit | 18 | 19 | 20 | 21 | 22 | 23 | 24 |
| Nonwoven | Synthetic | Materials | — | Acrylic | Acrylic | Acrylic | Acrylic | Acrylic | Acrylic | Acrylic |
| fabric | fibers | Single fiber | μm | 9 | 9 | 9 | 9 | 9 | 9 | 9 |
| composition | | diameter | | | | | | | | |
| | | Variation in | % | 8 | 8 | 8 | 8 | 8 | 9 | 10 |
| | | fiber diameter | | | | | | | | |
| | | Flatness | — | 1.2 | 1.2 | 1.2 | 1.2 | 1.2 | 1.2 | 1.2 |
| | | Recess depth | % | 5.6 | 5.6 | 5.6 | 5.6 | 5.6 | 5.6 | 5.6 |
| | | ratio | | | | | | | | |
| | | Tensile | — | 3.0 | 3.0 | 3.0 | 3.0 | 3.0 | 3.0 | 3.0 |
| | | strength | | | | | | | | |
| | | Content | Mass % | 58 | 58 | 52 | 32 | 52 | 58 | 58 |
| | Cellulose | Materials | — | Rayon | Rayon | Rayon | Rayon | Rayon | Rayon | Rayon |
| | fibers | Single fiber | μm | 12 | 12 | 12 | 12 | 12 | 12 | 12 |
| | | diameter | | | | | | | | |
| | | Variation in | % | 11 | 11 | 11 | 11 | 11 | 11 | 11 |
| | | fiber diameter | | | | | | | | |
| | | Content | Mass % | 42 | 42 | 48 | 68 | 48 | 42 | 42 |
| | Other | Materials | — | — | — | — | — | — | — | — |
| | Fibers | Fiber diameter | μm | — | — | — | — | — | — | — |
| | | Variation in | % | — | — | — | — | — | — | — |
| | | fiber diameter | | | | | | | | |
| | | Content | Mass % | — | — | — | — | — | — | — |
| | First | Content of | Mass % | 100 | 100 | 70 | 50 | 40 | 100 | 100 |
| | layer | synthetic | | | | | | | | |
| | | fiber | | | | | | | | |
| | | Content of | Mass % | 0 | 0 | 30 | 50 | 60 | 0 | 0 |
| | | cellulose | | | | | | | | |
| | | fiber | | | | | | | | |
| | Second | Content of | Mass % | 30 | 30 | 40 | 20 | 60 | 30 | 30 |
| | layer | synthetic | | | | | | | | |
| | | fiber | | | | | | | | |
| | | Content of | Mass % | 70 | 70 | 60 | 80 | 40 | 70 | 70 |
| | | cellulose | | | | | | | | |
| | | fiber | | | | | | | | |
| | | Content of | Mass % | 0 | 0 | 0 | 0 | 0 | 0 | 0 |
| | | other fibers | | | | | | | | |
| | | Average single | μm | 10 | 10 | 10 | 11 | 10 | 10 | 10 |
| | | fiber diameter | | | | | | | | |
| | | of all fibers | | | | | | | | |
| | | Variation in | % | 16 | 16 | 16 | 14 | 14 | 16 | 16 |
| | | fiber diameter | | | | | | | | |
| | | of all fibers | | | | | | | | |
| | Average single fiber | | μm | 10 | 10 | 10 | 10 | 10 | 10 | 10 |
| | diameter of all fibers | | | | | | | | | |
| | Basis | Total basis | g/cm² | 50 | 50 | 50 | 50 | 50 | 50 | 50 |
| | weight | weight | | | | | | | | |
| | | Basis weight of | g/cm² | 20 | 20 | 20 | 20 | 20 | 20 | 20 |
| | | first layer | | | | | | | | |
| | | Basis weight of | g/cm² | 30 | 30 | 30 | 30 | 30 | 30 | 30 |
| | | second layer | | | | | | | | |

| | | | | | | | | |
|---|---|---|---|---|---|---|---|---|
| Layer composition | — | 1/2 | 1/2 | 1/2 | 1/2 | 1/2 | 1/2 | 1/2 |

N6: nylon 6,
PET: polyethylene terephthalate

TABLE 5-2

| | | | | Example | Example | Example | Example | Example | Example | Example |
|---|---|---|---|---|---|---|---|---|---|---|
| | | | Unit | 25 | 26 | 27 | 28 | 29 | 30 | 31 |
| Nonwoven | Synthetic | Materials | — | Acrylic | Acrylic | Acrylic | Acrylic | Acrylic | Acrylic | Acrylic |
| fabric | fibers | Single fiber | μm | 9 | 9 | 9 | 9 | 9 | 9 | 9 |
| composition | | diameter | | | | | | | | |
| | | Variation in | % | 11 | 8 | 8 | 8 | 8 | 8 | 8 |
| | | fiber diameter | | | | | | | | |
| | | Flatness | — | 1.2 | 1.2 | 1.2 | 1.2 | 1.2 | 1.2 | 1.2 |
| | | Recess depth | % | 5.6 | 5.6 | 5.6 | 5.6 | 5.6 | 5.6 | 5.6 |
| | | ratio | | | | | | | | |
| | | Tensile | — | 3.0 | 3.0 | 3.0 | 3.0 | 3.0 | 3.0 | 3.0 |
| | | strength | | | | | | | | |
| | | Content | Mass % | 58 | 43 | 40 | 40 | 40 | 40 | 50 |
| | Cellulose | Materials | — | Rayon | Rayon | Rayon | Rayon | Rayon | Rayon | Rayon |
| | fibers | Single fiber | μm | 12 | 12 | 14 | 15 | 10 | 10 | 12 |
| | | diameter | | | | | | | | |
| | | Variation in | % | 11 | 11 | 11 | 11 | 11 | 11 | 11 |
| | | fiber diameter | | | | | | | | |
| | | Content | Mass % | 42 | 33 | 36 | 30 | 36 | 36 | 50 |
| | Other | Materials | — | — | PET | PET | PET | PET | PET | — |
| | Fibers | Fiber diameter | μm | — | 22 | 22 | 22 | 22 | 25 | — |
| | | Variation in | % | — | 13 | 13 | 13 | 13 | 13 | — |
| | | fiber diameter | | | | | | | | |
| | | Content | Mass % | — | 24 | 24 | 30 | 24 | 24 | — |
| | First | Content of | Mass % | 100 | 100 | 100 | 100 | 100 | 100 | 70 |
| | layer | synthetic | | | | | | | | |
| | | fiber | | | | | | | | |
| | | Content of | Mass % | 0 | 0 | 0 | 0 | 0 | 0 | 30 |
| | | cellulose | | | | | | | | |
| | | fiber | | | | | | | | |
| | Second | Content of | Mass % | 30 | 5 | 0 | 0 | 0 | 0 | 20 |
| | layer | synthetic | | | | | | | | |
| | | fiber | | | | | | | | |
| | | Content of | Mass % | 70 | 55 | 60 | 50 | 60 | 60 | 80 |
| | | cellulose | | | | | | | | |
| | | fiber | | | | | | | | |
| | | Content of | Mass % | 0 | 40 | 40 | 50 | 40 | 40 | 0 |
| | | other fibers | | | | | | | | |
| | | Average single | μm | 10 | 13 | 15 | 17 | 11 | 11 | 11 |
| | | fiber diameter | | | | | | | | |
| | | of all fibers | | | | | | | | |
| | | Variation in | % | 16 | 27 | 19 | 17 | 31 | 36 | 14 |
| | | fiber diameter | | | | | | | | |
| | | of all fibers | | | | | | | | |
| | Average single fiber | | μm | 10 | 12 | 12 | 12 | 11 | 11 | 10 |
| | diameter of all fibers | | | | | | | | | |
| | Basis | Total basis | g/cm² | 50 | 50 | 50 | 50 | 50 | 50 | 50 |
| | weight | weight | | | | | | | | |
| | | Basis weight of | g/cm² | 20 | 20 | 20 | 20 | 20 | 20 | 30 |
| | | first layer | | | | | | | | |
| | | Basis weight | g/cm² | 30 | 30 | 30 | 30 | 30 | 30 | 20 |
| | | of second | | | | | | | | |
| | | layer | | | | | | | | |

| | | | | | | | | |
|---|---|---|---|---|---|---|---|---|
| Layer composition | — | 1/2 | 1/2 | 1/2 | 1/2 | 1/2 | 1/2 | 1/2/1 |

N6: nylon 6,
PET: polyethylene terephthalate

TABLE 6-1

| | | | | | Example | Example | Example | Example | Example | Example | Example |
|---|---|---|---|---|---|---|---|---|---|---|---|
| | | | | Unit | 18 | 19 | 20 | 21 | 22 | 23 | 24 |
| Physical | Average coefficient of | | | — | 0.060 | 0.074 | 0.072 | 0.062 | 0.060 | 0.071 | 0.070 |
| properties | dynamic friction (MIU) | | | | | | | | | | |
| | Surface characteristics | | | — | 3.7 | 4.0 | 3.8 | 3.2 | 2.7 | 3.3 | 2.7 |
| | Strength at 10% elongation | | | N/25 mm | 1.1 | 1.6 | 1.9 | 2.7 | 2.9 | 1.8 | 1.8 |
| Monitor | First | Face | Adhesion | — | — | — | — | — | — | — | — |
| evaluation | time | mask | Soft touch | — | — | — | — | — | — | — | — |
| | | | to skin | | | | | | | | |
| | | Cleansing | Wiping | — | — | — | — | — | — | — | — |
| | | sheet | properties | | | | | | | | |
| | | | Soft touch | — | — | — | — | — | — | — | — |
| | | | to skin | | | | | | | | |
| | | | Handling | — | — | — | — | — | — | — | — |
| | | | properties | | | | | | | | |
| | | | during wiping | | | | | | | | |
| | Second | Face | Adhesion | — | B | A | A | B | C | A | A |
| | time | mask | Soft touch | — | A | A | A | B | C | B | C |
| | | | to skin | | | | | | | | |
| | | Cleansing | Wiping | — | B | A | A | B | C | A | A |
| | | sheet | properties | | | | | | | | |
| | | | Soft touch | — | A | A | A | B | C | B | C |
| | | | to skin | | | | | | | | |
| | | | Handling | — | C | A | A | A | A | A | A |
| | | | properties | | | | | | | | |
| | | | during wiping | | | | | | | | |

TABLE 6-2

| | | | | | Example | Example | Example | Example | Example | Example | Example |
|---|---|---|---|---|---|---|---|---|---|---|---|
| | | | | Unit | 25 | 26 | 27 | 28 | 29 | 30 | 31 |
| Physical | Average coefficient of | | | — | 0.070 | 0.068 | 0.063 | 0.061 | 0.061 | 0.060 | 0.076 |
| properties | dynamic friction (MIU) | | | | | | | | | | |
| | Surface characteristics | | | — | 2.4 | 3.4 | 2.8 | 2.3 | 2.8 | 2.3 | 4.0 |
| | Strength at 10% elongation | | | N/25 mm | 1.9 | 2.6 | 3.5 | 4.4 | 3.1 | 3.4 | 1.3 |
| Monitor | First | Face | Adhesion | — | — | — | — | — | — | — | — |
| evaluation | time | mask | Soft touch | — | — | — | — | — | — | — | — |
| | | | to skin | | | | | | | | |
| | | Cleansing | Wiping | — | — | — | — | — | — | — | — |
| | | sheet | properties | | | | | | | | |
| | | | Soft touch | — | — | — | — | — | — | — | — |
| | | | to skin | | | | | | | | |
| | | | Handling | — | — | — | — | — | — | — | — |
| | | | properties | | | | | | | | |
| | | | during wiping | | | | | | | | |
| | Second | Face | Adhesion | — | A | B | C | D | C | C | A |
| | time | mask | Soft touch | — | D | B | C | D | C | D | A |
| | | | to skin | | | | | | | | |
| | | Cleansing | Wiping | — | A | B | C | D | C | C | A |
| | | sheet | properties | | | | | | | | |
| | | | Soft touch | — | D | B | C | D | C | D | A |
| | | | to skin | | | | | | | | |
| | | | Handling | — | A | A | A | A | A | A | B |
| | | | properties | | | | | | | | |
| | | | during wiping | | | | | | | | |

### DESCRIPTION OF REFERENCE SIGNS

1: Cross section
2: Recess
3: Deepest point
4: Circumscribed circle
5: Contact point with circumscribed circle
6: Perpendicular line
N: Center point
L: Line segment

1. A nonwoven fabric for skin care products containing synthetic fibers and cellulose fibers, wherein
when a maximum length of a cross section of the synthetic fibers is denoted by A and a maximum width thereof is denoted by B, flatness expressed by the following formula (1) is 1.0 to 1.5:
flatness=A/B (1)
the synthetic fibers have recesses, a depth of which is 1.0% or more and 10.0% or less relative to a single fiber diameter of the synthetic fibers,
the single fiber diameter of the synthetic fibers is 1 μm or more and 20 μm or less,
a content of the synthetic fibers is 30 to 70% by mass relative to the entire nonwoven fabric for skin care products,
a content of the cellulose fibers is 70 to 30% by mass relative to the entire nonwoven fabric for skin care products, and
an average single fiber diameter of all fibers constituting the nonwoven fabric for skin care products is 5 μm or more and 20 μm or less.

2. The nonwoven fabric for skin care products according to claim 1, wherein the single fiber diameter of the synthetic fibers is 1 μm or more and 15 μm or less, and the average single fiber diameter of all fibers constituting the nonwoven fabric for skin care products is 5 μm or more and 15 μm or less.

3. The nonwoven fabric for skin care products according to claim 1, wherein a tensile strength of the synthetic fibers measured in accordance with JIS L1015 2010 8.7.1 is 2.5 to 3.2 cN/dtex.

4. The nonwoven fabric for skin care products according to claim 1, comprising a laminated nonwoven fabric structure including a first layer and a second layer, wherein
the first layer constitutes one surface of the nonwoven fabric for skin care products, the first layer contains the synthetic fibers in an amount of 50% by mass or more relative to the entire first layer, and a variation in the single fiber diameter of the synthetic fibers in the first layer is 1 to 10%,
the average single fiber diameter of fibers constituting the second layer is 5 to 15 μm, and a variation in the single fiber diameter of fibers constituting the second layer is 11 to 35%, and
fibers constituting the first layer and fibers constituting the second layer are entangled.

5. A face mask comprising the nonwoven fabric for skin care products according to claim 1.

6. A cleansing sheet comprising the nonwoven fabric for skin care products according to claim 1.
